(19) United States
(12) Patent Application Publication
(10) Pub. No.: US 2026/0167982 A1
(43) Pub. Date: Jun. 18, 2026

(54) INSECTICIDAL PROTEINS COMPOSITIONS AND METHODS OF USE

(71) Applicant: GENECTIVE SA, Champaign, IL (US)

(72) Inventors: James Winsor, Champaign, IL (US); Yuanyuan Hu, Champaign, IL (US); Madison Havel, Champaign, IL (US); Erik Schad, Champaign, IL (US); Laurent Décousset, Champaign, IL (US); Lainey Williamson, Champaign, IL (US); Mickael Bosio, Champaign, IL (US); Christophe Sallaud, Champaign, IL (US)

(21) Appl. No.: 19/418,281

(22) Filed: Dec. 12, 2025

Related U.S. Application Data
(63) Continuation of application No. PCT/US2025/048776, filed on Sep. 30, 2025.
(60) Provisional application No. 63/703,495, filed on Oct. 4, 2024.

(51) Int. Cl. C12N 15/82

(52) U.S. Cl. CPC C12N 15/8261

(57) ABSTRACT

Disclosed herein are transformed plants, plant tissues, plant parts, plant cells, and plant seeds comprising a recombinant nucleic acid molecule encoding a polypeptide having pesticidal activity. Also disclosed herein are methods of protecting or treating a plant from infection by a plant pathogen or pest by transforming plants, plant tissues, plant parts, plant cells, and plant seeds with a recombinant nucleic acid molecule encoding a polypeptide having pesticidal activity.

### CROSS-REFERENCE TO RELATED APPLICATIONS

This application is a continuation of International Patent Application No. PCT/US2025/048776, filed on Sep. 30, 2025, which claims priority to U.S. Provisional Patent Application No. 63/703,495, filed on Oct. 4, 2024, the entire contents of each of which are fully incorporated herein by reference.

### REFERENCE TO SEQUENCE LISTING

This application was filed with a Sequence Listing XML in ST.26 XML format in accordance with 37 C.F.R. § 1.831 and PCT Rule 13ter. The Sequence Listing XML file submitted in the USPTO Patent Center, “218903-0046-WO01_GEN00021WOPCT_Sequence Listing.xml,” was created on Sep. 17, 2025, contains 56 sequences, has a file size of 64.0 Kbytes (65,536 bytes), and is incorporated by reference in its entirety into the specification.

### FIELD

This disclosure relates to the field of molecular biology, specifically, novel genes and engineered genes that encode pesticidal proteins useful for controlling pathogens and pests, particularly plant pests. These proteins and the nucleic acid sequences that encode them are useful in preparing pesticidal compositions and in the production of transgenic pest-resistant plants. The disclosure also relates generally to compositions and methods for controlling pathogens and pests, particularly plant pests.

### INTRODUCTION

Across the world, crops are subjected to multiple threats e.g., pests, plant diseases, and weeds. Losses due to pests and diseases are greatly threatening global food supply, hence the necessity to develop solutions to avoid partial or complete destruction of cultures. The main solutions are chemicals, biocontrols, or genetically modified organisms.

Current strategies use genes expressing pesticidal proteins to produce transgenic crops. These pesticidal proteins are generally derived from Bacillus thuringiensis (“Bt”), a Gram-positive spore forming soil bacterium. Current commercial pesticidal proteins include Bt Cry (crystal protein), or VIP (Vegetative Insecticidal Protein). Transgenic crops expressing insecticidal proteins are used to combat crop damage from insects.

The wide adoption of pesticidal protein-based technologies by farmers for controlling insects in the fields gave rise to resistance to these pesticidal proteins in some target pests in many parts of the world. One way of solving this problem is stacking pesticidal protein genes with different modes of action against insects in transformed plants. In order to find new pesticidal proteins with new modes of action, possible strategies involve discovering new pesticidal proteins from new sources or protein engineering. Thus, there is a need for novel insecticidal proteins for controlling plant pests.

### SUMMARY

In one aspect, the disclosure relates to a method of protecting a plant from infection by a plant pathogen or pest, the method comprising: transforming the plant with a nucleic acid molecule encoding a polypeptide having at least 80% sequence identity to any one of SEQ ID NOs: 1-56 to generate a transformed plant expressing the polypeptide, wherein said polypeptide has pesticidal activity against the plant pathogen or pest; and regenerating the transformed plant expressing the polypeptide. In an embodiment, the polypeptide has at least 95% sequence identity to any one of SEQ ID NOs: 1-56. In another embodiment, the polypeptide is any one of SEQ ID NOs: 1-56. In another embodiment, the plant pathogen or pest is selected from the group consisting of fall armyworm (Spodoptera frugiperda), corn earworm (Helicoverpa zea), European corn borer (Ostrinia nubilalis), cotton boll worm (Helicoverpa armigera), black cutworm (Agrotis ipsilon), lesser cornstalk borer (Elasmopalpus lignosellus), Asian corn borer (Ostinia furnacalis), southwestern corn borer (Diatraea grandiosella), sugarcane borer (Diatraea saccharalis), western bean cutworm (Striacosta albicosta), velvetbean caterpillar (Anticarsia gemmatalis), corn rootworm (Diabrotica virgifera), southern corn rootworm (Diabrotica undecimpunctata howardi), northern corn rootworm (Diabrotica barberi), soybean looper (Chrysodeixis includens), tobacco budworm (Chloridia virescens), beet armyworm (Spodoptera exigua), southern armyworm (Spodoptera eridania), and combinations thereof.

In a further aspect, the disclosure relates to a transformed plant, seed, or plant part comprising a recombinant nucleic acid molecule encoding a polypeptide having at least 80% sequence identity to any one of SEQ ID NOs: 1-56 stably incorporated into a genome of the transformed plant, seed, or plant part, wherein the transformed plant, seed, or plant part stably expresses the polypeptide, and wherein the polypeptide has pesticidal activity against a plant pathogen or pest. In an embodiment, the polypeptide has at least 95% sequence identity to any one of SEQ ID NOs: 1-56. In another embodiment, the polypeptide is any one of SEQ ID NOs: 1-56. In another embodiment, the transformed plant, seed, or plant part is selected from the group consisting of rice, barley, sorghum, soybean, cotton, maize, rapeseed, sugar cane, tobacco, sunflower, and wheat.

Another aspect of the disclosure provides a recombinant nucleic acid molecule comprising a polynucleotide sequence encoding a polypeptide having at least 80% sequence identity to any one of SEQ ID NOs: 1-56, wherein the polypeptide has pesticidal activity against a plant pathogen or pest. In an embodiment, the polypeptide has at least 95% sequence identity to any one of SEQ ID NOs: 1-56. In another embodiment, the polypeptide is any one of SEQ ID NOs: 1-56. In another embodiment, the polynucleotide sequence encoding the polypeptide is operably linked to one or more promoter sequences.

Another aspect of the disclosure provides a vector comprising a recombinant nucleic acid molecule comprising a polynucleotide sequence encoding a polypeptide having at least 80% sequence identity to any one of SEQ ID NOs: 1-56, wherein the polypeptide has pesticidal activity against a plant pathogen or pest. In an embodiment, the polypeptide has at least 95% sequence identity to any one of SEQ ID NOs: 1-56. In another embodiment, the polypeptide is any one of SEQ ID NOs: 1-56.

Another aspect of the disclosure provides a transformed host cell comprising a recombinant nucleic acid molecule comprising a polynucleotide sequence encoding a polypeptide having at least 80% sequence identity to any one of SEQ ID NOs: 1-56, wherein the polypeptide has pesticidal activity against a plant pathogen or pest. In an embodiment, the polypeptide has at least 95% sequence identity to any one of SEQ ID NOs: 1-56. In another embodiment, the polypeptide is any one of SEQ ID NOs: 1-56.

Another aspect of the disclosure provides a method of treating a plant or plant part against a plant pathogen or pest, the method comprising: applying to the plant or plant part an effective amount of at least one polypeptide having at least 80% sequence identity to any one of SEQ ID NOs: 1-56, wherein the polypeptide has pesticidal activity against the plant pathogen or pest. In an embodiment, the polypeptide has at least 95% sequence identity to any one of SEQ ID NOs: 1-56. In another embodiment, the polypeptide is any one of SEQ ID NOs: 1-56.

Another aspect of the disclosure provides a composition having insecticidal activity against a plant pathogen or pest, the composition comprising an effective amount of at least one polypeptide having at least 80% sequence identity to any one of SEQ ID NOs: 1-56. In an embodiment, the polypeptide has at least 95% sequence identity to any one of SEQ ID NOs: 1-56. In another embodiment, the polypeptide is anyone of SEQ ID NOs: 1-56.

Another aspect of the disclosure provides a composition having insecticidal activity against a plant pathogen or pest, the composition comprising a polynucleotide encoding at least one polypeptide having at least 80% sequence identity to any one of SEQ ID NOs: 1-56. In an embodiment, the polypeptide has at least 95% sequence identity to any one of SEQ ID NOs: 1-56. In an embodiment, the polypeptide is any one of SEQ ID NOs: 1-56.

Another aspect of the disclosure provides polynucleotides comprising a polynucleotide sequence encoding any of the insecticidal polypeptides described herein operably linked to a heterologous regulatory element.

Another aspect of the disclosure provides cells comprising any of the polynucleotides described herein. In an embodiment, the cell is a plant cell or a bacteria cell.

Another aspect of the disclosure provides modified plants comprising any of the polynucleotides or cells described herein.

Another aspect of the disclosure provides compositions and methods for modifying bacteria, plants, plant cells, tissues, and seeds to provide insect resistance. In some embodiments, nucleic acid molecules encode sequences for pesticidal and insecticidal polypeptides, vectors comprise those nucleic acid molecules, and host cells comprise the vectors. Compositions may also include the pesticidal polypeptide sequences and antibodies to those polypeptides. Compositions may also comprise modified bacteria, plants, plant cells, tissues, and seeds.

This disclosure provides for other aspects and embodiments that will be apparent considering the following detailed description and accompanying figures.

### BRIEF DESCRIPTION OF THE DRAWINGS

FIG. 1 is a table of sequence descriptions for SEQ ID NOs: 1-56.

FIG. 2 is a depiction of a sequence alignment between the disclosed insecticidal proteins of SEQ ID NOs: 1-34.

FIG. 3 is homology tables showing amino acid sequence identity for the disclosed insecticidal proteins of SEQ ID NOs: 1-56.

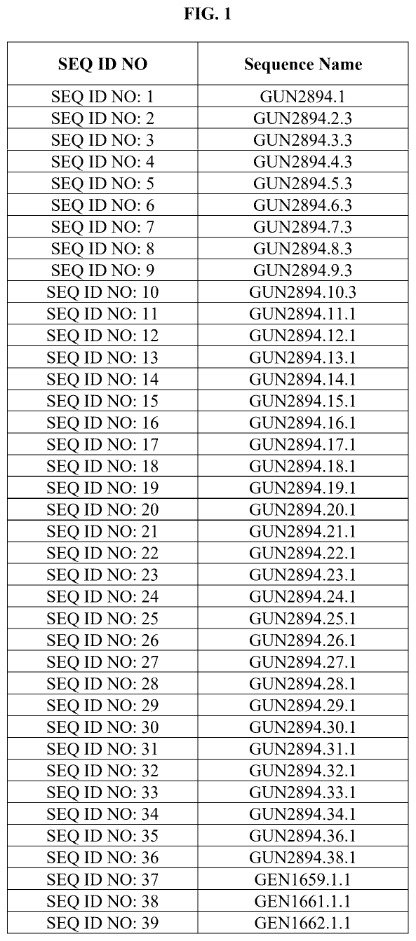

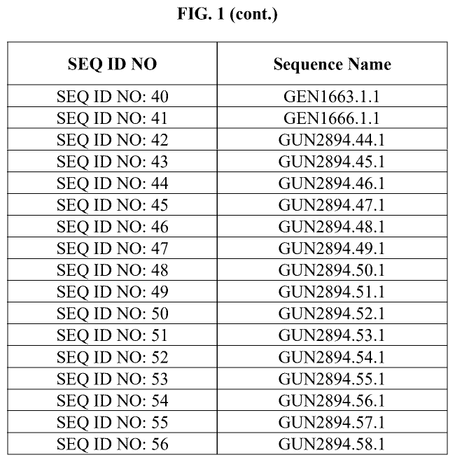

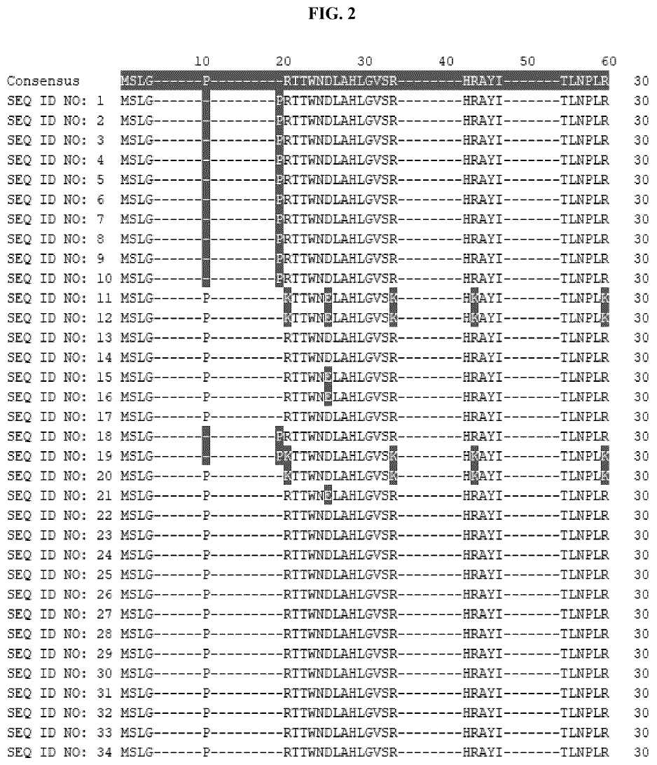

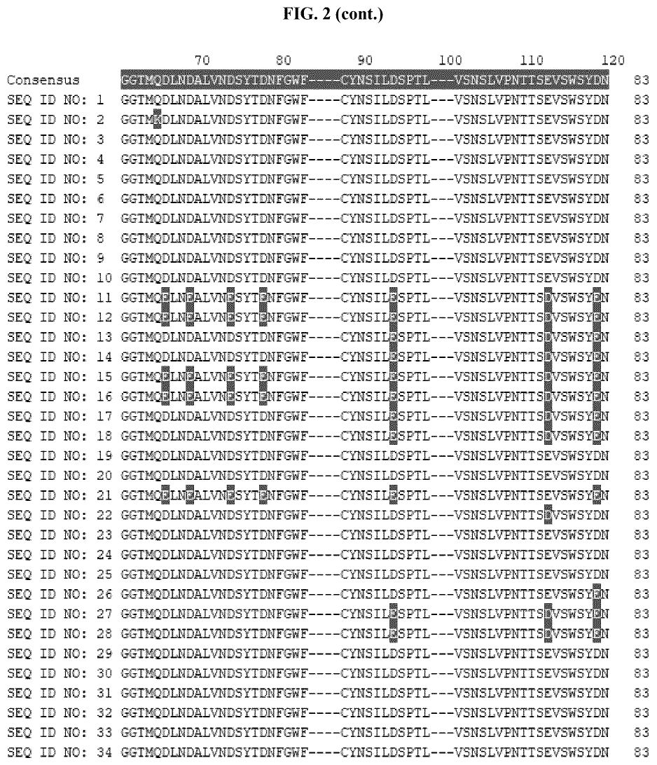

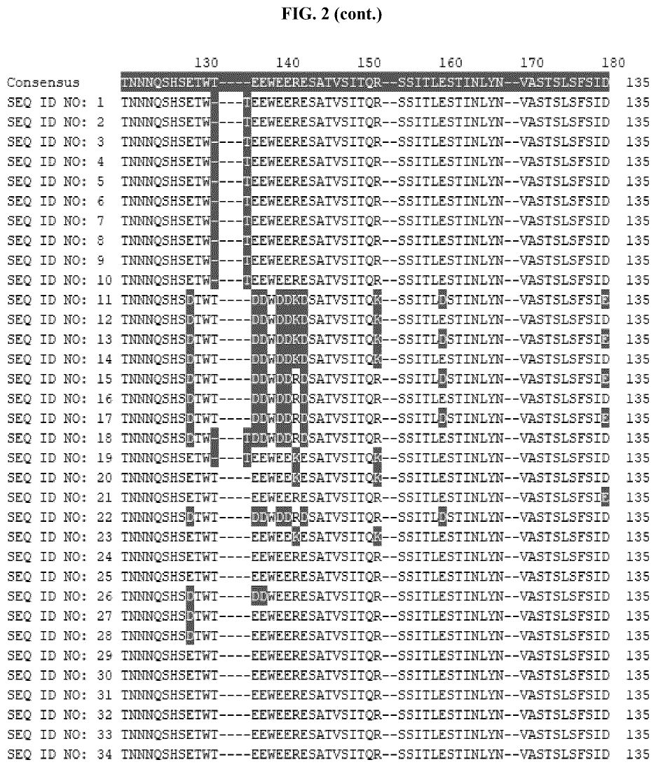

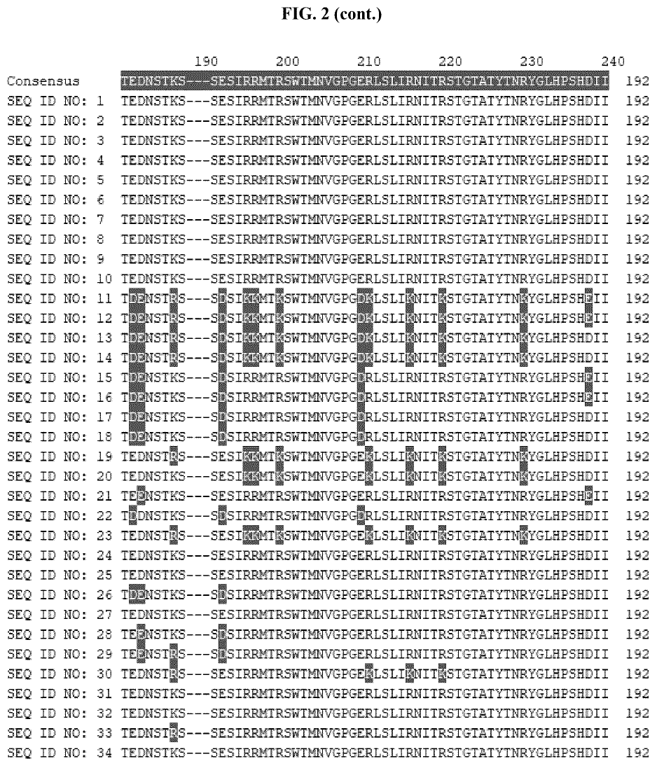

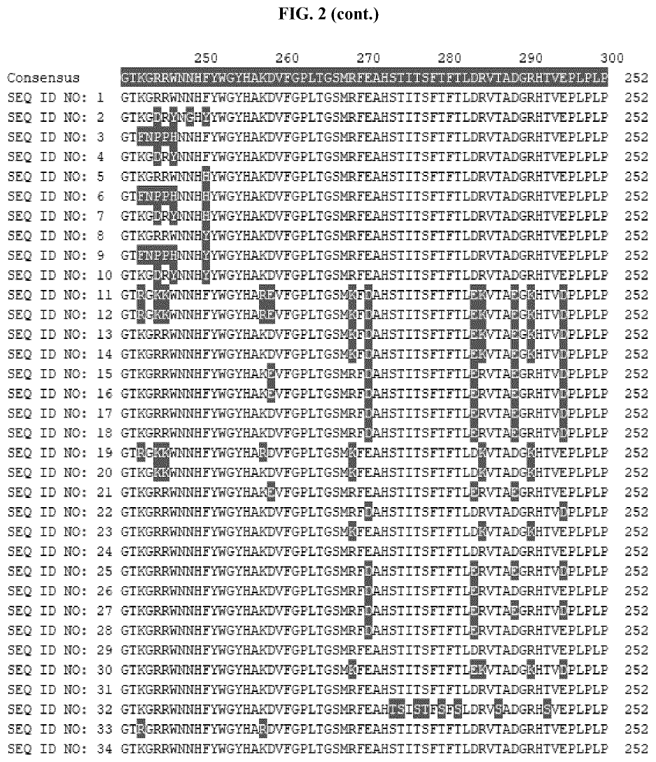

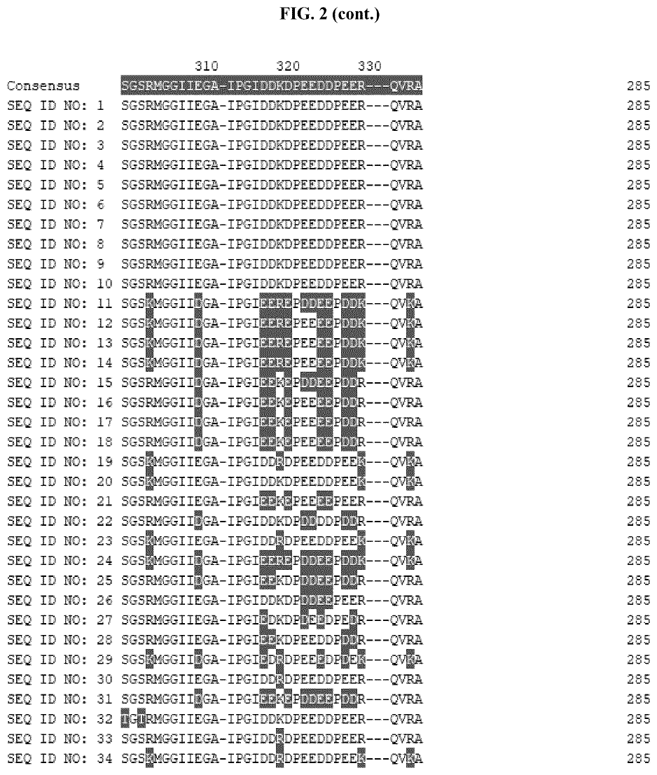

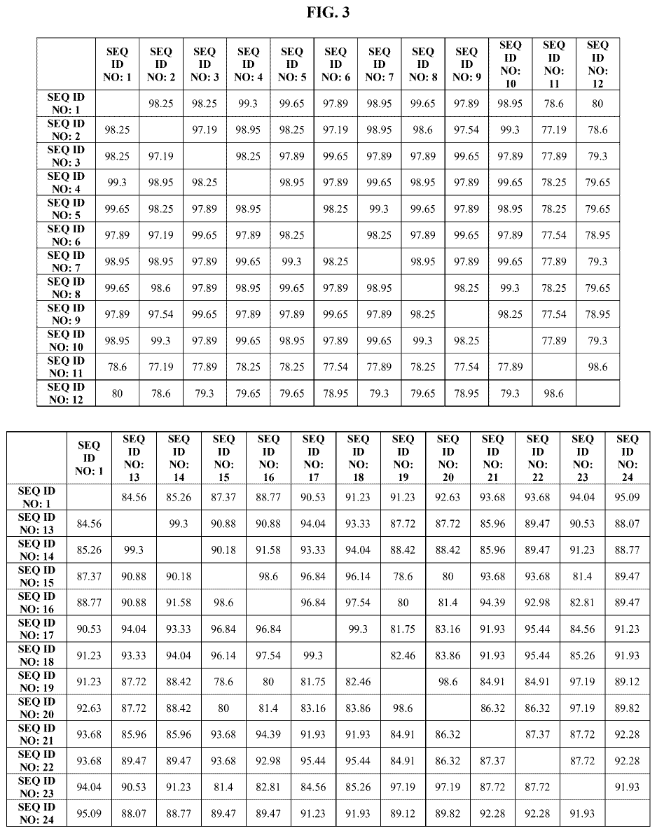

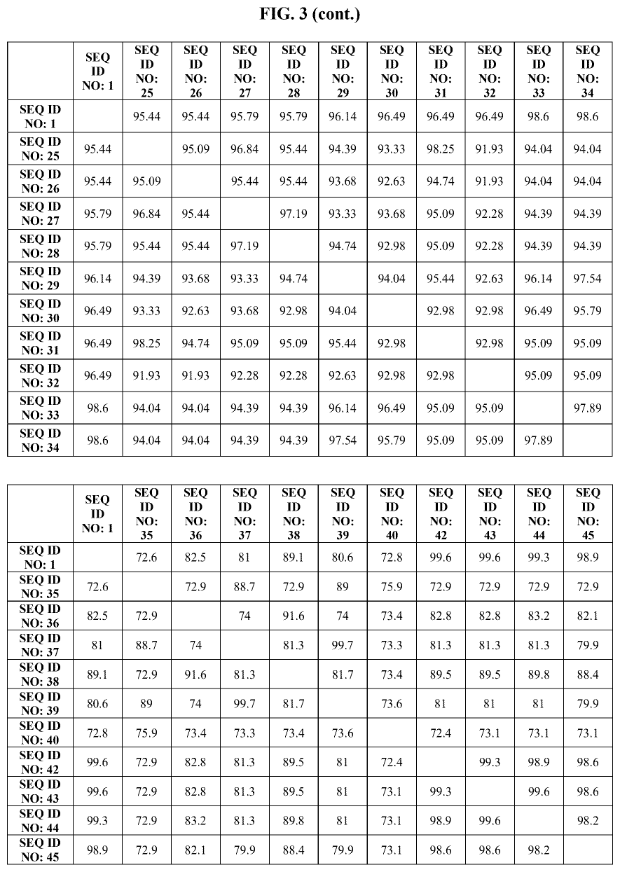

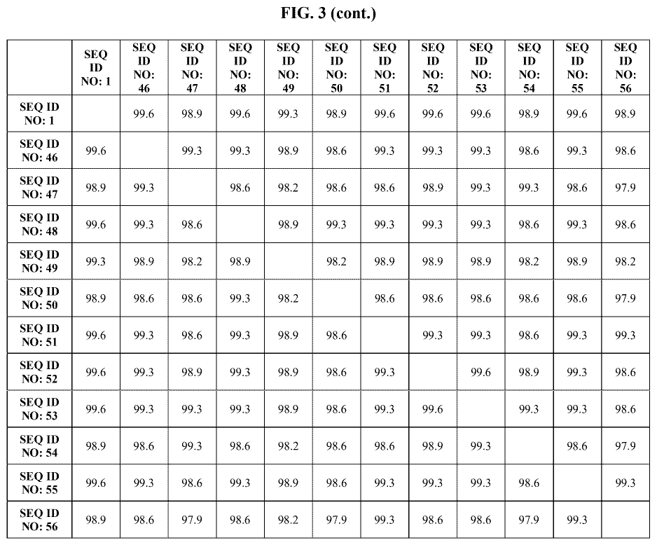

Before any embodiments of this disclosure are explained in detail, it is to be understood that the disclosure is not limited in its application to the details of construction and the arrangement of components set forth in the following description or illustrated in the accompanying figures. Also, it is to be understood that the phraseology and terminology used herein is for the purpose of description and should not be regarded as limiting.

### DETAILED DESCRIPTION

Described herein are compositions and methods comprising insecticidal proteins useful for conferring pesticidal activity. Disclosed compositions may include isolated, recombinant, and purified polypeptides having pesticidal activity. In some embodiments, recombinant nucleic acid molecules including DNA constructs and vectors that encode polypeptides having pesticidal activity are described herein. In some embodiments, nucleic acid molecules and polypeptides may be described as DNA constructs and expression cassettes for transforming plants, plant tissues, plant parts, plant cells, and plant seeds, as well as microorganisms. Polypeptides having pesticidal activity as described herein may provide useful alternatives to those currently deployed in commercial transgenic plants.

In some embodiments, the compositions and methods disclosed herein may comprise engineered Cry-like or Mpp-like proteins having pesticidal activity. In other embodiments, the compositions and methods may comprise engineered proteins having pesticidal activity that do not have any significant sequence identity with other known pesticidal proteins.

Unless otherwise defined herein, all technical and scientific terms used in connection with the present disclosure shall have the same meanings that are commonly understood by those of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting. Further, unless otherwise required by context, singular terms shall include pluralities and plural terms shall include the singular.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “about” or “approximately” as used herein as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In certain aspects, the term “about” refers to a range of values that fall within 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Alternatively, “about” can mean within 3 or more than 3 standard deviations, per the practice in the art. Alternatively, such as with respect to biological systems or processes, the term “about” can mean within an order of magnitude, preferably within 5-fold, and more preferably within 2-fold, of a value.

“Amino acid” as used herein refers to naturally occurring and non-natural synthetic amino acids, as well as amino acid analogs and amino acid mimetics that function in a manner similar to the naturally occurring amino acids. Naturally occurring amino acids are those encoded by the genetic code. Amino acids can be referred to herein by either their commonly known three-letter symbols or by the one-letter symbols recommended by the IUPAC-IUB Biochemical Nomenclature Commission. Amino acids include the side chain and polypeptide backbone portions.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an organism to which the nucleic acid is administered. The coding sequence may be codon optimized.

“Complement” or “complementary” as used herein can mean Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

The terms “control,” “reference level,” and “reference” are used herein interchangeably. The reference level may be a predetermined value or range, which is employed as a benchmark against which to assess the measured result. “Control group” as used herein refers to a group of control organisms. The predetermined level may be a cutoff value from a control group. The predetermined level may be an average from a control group. The normal levels or ranges for a target or for a protein activity may be defined in accordance with standard practice. A control may be an organism or cell without a vector as detailed herein. A control may be an organism, or a sample therefrom, whose condition is known. The organism, or sample therefrom, may be healthy, exposed to a toxin, exposed to a toxin prior to treatment, exposed to a toxin during treatment, or exposed to a toxin after treatment, or a combination thereof.

“Derived” and “derived from” as used herein refers to a DNA or amino acid sequence or a part of a DNA or amino acid sequence that has part or all of the sequence found in a native gene or protein.

“Functional” and “full-functional” as used herein describes protein that has biological activity. A “functional gene” refers to a gene transcribed to mRNA, which is translated to a functional protein.

“Fusion protein” as used herein refers to a chimeric protein created through the joining of two or more genes or gene fragments that originally coded for separate polypeptides. The translation of the fusion gene results in a single polypeptide with functional properties derived from each of the original polypeptides. A “chimeric protein” as used herein refers to a polypeptide comprising at least one polypeptide segment from two heterologous genes or two heterologous polypeptides.

“Genetic construct” or “construct” as used herein refers to the DNA or RNA nucleic acid molecules that comprise a polynucleotide that encodes a protein. The coding sequence includes initiation and termination signals operably linked to regulatory elements including a promoter and polyadenylation signal capable of directing expression in the cells of the organism to which the nucleic acid molecule is administered. As used herein, the term “expressible form” refers to gene constructs that contain the necessary regulatory elements operable linked to a coding sequence that encodes a protein such that when present in the cell of the organism, the coding sequence will be expressed.

The term “heterologous” as used herein refers to nucleic acid comprising two or more subsequences that are not found in the same relationship to each other in nature. For instance, a nucleic acid that is recombinantly produced typically has two or more sequences from unrelated genes synthetically arranged to make a new functional nucleic acid, for example, a promoter from one source and a coding region from another source. The two nucleic acids are thus heterologous to each other in this context. When added to a cell, the recombinant nucleic acids would also be heterologous to the endogenous genes of the cell. Thus, in a chromosome, a heterologous nucleic acid would include a non-native (non-naturally occurring) nucleic acid that has integrated into the chromosome, or a non-native (non-naturally occurring) extrachromosomal nucleic acid. Similarly, a heterologous protein indicates that the protein comprises two or more subsequences that are not found in the same relationship to each other in nature (for example, a “fusion protein,” where the two subsequences are encoded by a single nucleic acid sequence). A heterologous polynucleotide may be created using any gene editing or molecular biological technique. As used herein, a “heterologous domain” refers to a protein domain region that is combined with one or more naturally occurring domain regions to form a non-native (non-naturally occurring) engineered fusion protein, where the heterologous domain and the one or more naturally occurring domain regions are not found in the same relationship to each other in nature.

“Identical” or “identity” as used herein in the context of two or more polynucleotide or polypeptide sequences means that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of a single sequence are included in the denominator but not the numerator of the calculation. When comparing DNA and RNA, thymine (T) and uracil (U) may be considered equivalent. Determining the percent sequence identity between any two or more nucleic acid or amino acid sequences can be accomplished using one or more mathematical algorithms. For example, identity may be performed manually or by using a computer sequence algorithm such as BLAST or BLAST 2.0.

“Natural gene” as used herein refers to a gene that has not undergone a change, such as a loss, gain, or exchange of genetic material. The natural gene undergoes normal gene transmission and gene expression. For example, a natural gene may be a wild-type (i.e., native) gene.

“Nucleic acid” or “oligonucleotide” or “polynucleotide” as used herein means at least two nucleotides covalently linked together. The depiction of a single strand also defines the sequence of the complementary strand. Thus, a polynucleotide also encompasses the complementary strand of a depicted single strand. Many variants of a polynucleotide may be used for the same purpose as a given polynucleotide. Thus, a polynucleotide also encompasses substantially identical polynucleotides and complements thereof. A single strand provides a probe that may hybridize to a target sequence under stringent hybridization conditions. Thus, a polynucleotide also encompasses a probe that hybridizes under stringent hybridization conditions. Polynucleotides may be single stranded or double stranded or may contain portions of both double stranded and single stranded sequence. The polynucleotide can be nucleic acid, natural or synthetic, DNA, genomic DNA, cDNA, RNA, or a hybrid, where the polynucleotide can contain combinations of deoxyribo- and ribo-nucleotides, and combinations of bases including, for example, uracil, adenine, thymine, cytosine, guanine, inosine, xanthine hypoxanthine, isocytosine, and isoguanine. Polynucleotides can be obtained by chemical synthesis methods or by recombinant methods.

“Open reading frame” refers to a stretch of codons that begins with a start codon and ends at a stop codon. In eukaryotic genes with multiple exons, introns are removed, and exons are then joined together after transcription to yield the final mRNA for protein translation. An open reading frame may be a continuous stretch of codons. In some embodiments, the open reading frame only applies to spliced mRNAs, not genomic DNA, for expression of a protein.

“Operably linked” as used herein means that expression of a gene is under the control of a or influenced by a regulatory element (e.g., promoter) with which it is spatially connected. A regulatory element may be positioned 5′ (upstream) or 3′ (downstream) of a gene. The distance between a regulatory element and a gene may be approximately the same as the distance between that regulatory element and the gene it controls in the gene from which the regulatory element is derived. Variation in this distance may be accommodated without loss of regulatory function. Nucleic acid or amino acid sequences are “operably linked” (or “operatively linked”) when placed into a functional relationship with one another. For instance, a regulatory element is operably linked to a coding sequence if it regulates, or contributes to the modulation of, the transcription of the coding sequence. Operably linked DNA sequences are typically contiguous, and operably linked amino acid sequences are typically contiguous and in the same reading frame. However, since enhancers generally function when separated from the promoter by up to several kilobases or more and intronic sequences may be of variable lengths, some polynucleotide elements may be operably linked but not contiguous. Similarly, certain amino acid sequences that are non-contiguous in a primary polypeptide sequence may nonetheless be operably linked due to, for example folding of a polypeptide chain. With respect to fusion polypeptides, the terms “operatively linked” and “operably linked” can refer to the fact that each of the components performs the same function in linkage to the other component as it would if it were not so linked.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and transport proteins. The terms “polypeptide”, “protein,” and “peptide” are used interchangeably herein. “Primary structure” refers to the amino acid sequence of a particular peptide. “Secondary structure” refers to locally ordered, three dimensional structures within a polypeptide. These structures are commonly known as domains, for example, enzymatic domains, extracellular domains, transmembrane domains, pore domains, and cytoplasmic tail domains. “Domains” are portions of a polypeptide that form a compact unit of the polypeptide and are typically 15 to 350 amino acids long. Exemplary domains include domains with enzymatic activity or ligand binding activity. Typical domains are made up of sections of lesser organization such as stretches of beta-sheet and alpha-helices. “Tertiary structure” refers to the complete three-dimensional structure of a polypeptide monomer. “Quaternary structure” refers to the three-dimensional structure formed by the noncovalent association of independent tertiary units. A “motif” is a portion of a polypeptide sequence and includes at least two amino acids. A motif may be 2 to 20, 2 to 15, or 2 to 10 amino acids in length. A domain may be comprised of a series of the same type of motif.

“Pest” includes, but is not limited to, insects, fungi, bacteria, nematodes, mites, ticks, and the like. Insect pests may include insects selected from the orders Coleoptera, Diptera, Hymenoptera, Lepidoptera, Mallophaga, Homoptera, Hemiptera, Orthroptera, Thysanoptera, Dermaptera, Isoptera, Anoplura, Siphonaptera, and Trichoptera.

In certain embodiments described herein, insect pests may include larvae and adults of the order Coleoptera including weevils from the families Anthribidae, Bruchidae, and Curculionidae including, but not limited to: Anthonomus grandis Boheman (boll weevil); Cylindrocopturus adspersus LeConte (sunflower stem weevil); Diaprepes abbreviatus Linnaeus (Diaprepes root weevil); Hypera punctata Fabricius (clover leaf weevil); Lissorhoptrus oryzophilus Kuschel (rice water weevil); Metamasius hemipterus hemipterus Linnaeus (West Indian cane weevil); M. hemipterus sericeus Olivier (silky cane weevil); Sitophilus granarius Linnaeus (granary weevil); S. oryzae Linnaeus (rice weevil); Smicronyx fulvus LeConte (red sunflower seed weevil); S. sordidus LeConte (gray sunflower seed weevil); Sphenophorus maidis Chittenden (maize billbug); Rhabdoscelus obscurus Boisduval (New Guinea sugarcane weevil); flea beetles, cucumber beetles, rootworms, leaf beetles, potato beetles, and leafminers in the family Chrysomelidae including, but not limited to: Chaetocnema ectypa Horn (desert corn flea beetle); C. pulicaria Melsheimer (corn flea beetle); Colaspis brunnea Fabricius (grape colaspis); Diabrotica barberi Smith & Lawrence (northern corn rootworm); D. undecimpunctata howardi Barber (southern corn rootworm); D. virgifera virgifera LeConte (western corn rootworm); Leptinotarsa decemlineata Say (Colorado potato beetle); Oulema melanopus Linnaeus (cereal leaf beetle); Phyllotreta cruciferae Goeze (corn flea beetle); Zygogramma exclamationis Fabricius (sunflower beetle); beetles from the family Coccinellidae including, but not limited to: Epilachna varivestis Mulsant (Mexican bean beetle); chafers and other beetles from the family Scarabaeidae including, but not limited to: Antitrogus parvulus Britton (Childers cane grub); Cyclocephala borealis Arrow (northern masked chafer, white grub); C. immaculata Olivier (southern masked chafer, white grub); Dermolepida albohirtum Waterhouse (Greyback cane beetle); Euetheola humilis rugiceps LeConte (sugarcane beetle); Lepidiota frenchi Blackburn (French's cane grub); Tomarus gibbosus De Geer (carrot beetle); T. subtropicus Blatchley (sugarcane grub); Phyllophaga crinita Burmeister (white grub); P. latifrons LeConte (June beetle); Popillia japonica Newman (Japanese beetle); Rhizotrogus majalis Razoumowsky (European chafer); carpet beetles from the family Dermestidae; wireworms from the family Elateridae, Eleodes spp., Melanotus spp. including M. communis Gyllenhal
(wireworm); Conoderus spp.; Limonius spp.; Agriotes spp.; Ctenicera spp.; Aeolus spp.; bark beetles from the family Scolytidae; beetles from the family Tenebrionidae; beetles from the family Cerambycidae such as, but not limited to, Migdolus fryanus Westwood (longhorn beetle); and beetles from the Buprestidae family including, but not limited to, Aphanisticus cochinchinae seminulum Obenberger (leaf-mining buprestid beetle).

In other embodiments, insect pests may include immatures and adults of the order Diptera, including leafminers Agromyza parvicornis Loew (corn blotch leafminer); midges including, but not limited to: Contarinia sorghicola Coquillett (sorghum midge); Mayetiola destructor Say (Hessian fly); Neolasioptera murtfeldtiana Felt, (sunflower seed midge); Sitodiplosis mosellana Géhin (wheat midge); fruit flies (Tephritidae), Oscinella frit Linnaeus (frit flies); maggots including, but not limited to: Delia spp. including Delia platura Meigen (seedcorn maggot); D. coarctata Fallen (wheat bulb fly); Fannia canicularis Linnaeus, F. femoralis Stein (lesser house flies); Meromyza americana Fitch (wheat stem maggot); Musca domestica Linnaeus (house flies); Stomoxys calcitrans Linnaeus (stable flies)); face flies, horn flies, blow flies, Chrysomya spp.; Phormia spp.; and other muscoid fly pests, horse flies Tabanus spp.; bot flies Gastrophilus spp.; Oestrus spp.; cattle grubs Hypoderma spp.; deer flies Chrysops spp.; Melophagus ovinus Linnaeus (keds); and other Brachycera, mosquitoes Aedes spp.; Anopheles spp.; Culex spp.; black flies Prosimulium spp.; Simulium spp.; biting midges, sand flies, sciarids, and other Nematocera.

Lepidoptera insects may include, but are not limited to, armyworms, cutworms, loopers, and heliothines in the family Noctuidae: Agrotis ipsilon Hufnagel (black cutworm); A. orthogonia Morrison (western cutworm); A. segetum Denis & Schiffermüller (turnip moth); A. subterranea Fabricius (granulate cutworm); Alabama argillacea Hubner (cotton leaf worm); Anticarsia gemmatalis Hubner (velvetbean caterpillar); Athetis mindara Barnes and McDunnough (rough skinned cutworm); Earias insulana Boisduval (spiny bollworm); E. vittella Fabricius (spotted bollworm); Egira (Xylomyges) curialis Grote (citrus cutworm); Euxoa messoria Harris (darksided cutworm); Helicoverpa armigera Hubner (American bollworm); H. zea Boddie (corn earworm or cotton bollworm); Heliothis virescens Fabricius (tobacco budworm); Hypena scabra Fabricius (green cloverworm); Mamestra configurata Walker (bertha armyworm); M. brassicae Linnaeus (cabbage moth); Melanchra picta Harris (zebra caterpillar); Pseudaletia unipuncta Haworth (armyworm); Pseudoplusia includens Walker (soybean looper); Richia albicosta Smith (Western bean cutworm); Spodoptera frugiperda JE Smith (fall armyworm); S. exigua Hubner (beet armyworm); S. litura Fabricius (tobacco cutworm, cluster caterpillar); Trichoplusia ni Hubner (cabbage looper); borers, casebearers, webworms, coneworms, and skeletonizers from the families Pyralidae and Crambidae such as Achroia grisella Fabricius (lesser wax moth); Amyelois transitella Walker (naval orangeworm); Anagasta kuehniella Zeller (Mediterranean flour moth); Cadra cautella Walker (almond moth); Chilo partellus Swinhoe (spotted stalk borer); C. suppressalis Walker (striped stem/rice borer); C. terrenellus Pagenstecher (sugarcane stemp borer); Corcyra cephalonica Stainton (rice moth); Crambus caliginosellus Clemens (corn root webworm); C. teterrellus Zincken (bluegrass webworm); Cnaphalocrocis medinalis Guenee (rice leaf roller); Desmia funeralis Hubner (grape leaffolder); Diaphania hyalinata Linnaeus (melon worm); D. nitidalis Stoll (pickleworm); Diatraea grandiosella Dyar (southwestern corn borer), D. saccharalis Fabricius (surgarcane borer); Elasmopalpus lignosellus Zeller (lesser cornstalk borer); Eoreuma loftini Dyar (Mexican rice borer); Ephestia elutella Hubner (tobacco (cacao) moth); Galleria mellonella Linnaeus (greater wax moth); Hedylepta accepta Butler (sugarcane leafroller); Herpetogramma licarsisalis Walker (sod webworm); Homoeosoma electellum Hulst (sunflower moth); Loxostege sticticalis Linnaeus (beet webworm); Maruca testulalis Geyer (bean pod borer); Orthaga thyrisalis Walker (tea tree web moth); Ostrinia nubilalis Hubner (European corn borer); Plodia interpunctella Hubner (Indian meal moth); Scirpophaga incertulas Walker (yellow stem borer); Udea rubigalis Guenee (celery leaftier); and leafrollers, budworms, seed worms, and fruit worms in the family Tortricidae Acleris gloverana Walsingham (Western blackheaded budworm); A. variana Fernald (Eastern blackheaded budworm); Adoxophyes orana Fischer von Rösslerstamm (summer fruit tortrix moth); Archips spp. including A. argyrospila Walker (fruit tree leaf roller) and A. rosana Linnaeus (European leaf roller); Argyrotaenia spp.; Bonagota salubricola Meyrick (Brazilian apple leafroller); Choristoneura spp.; Cochylis hospes Walsingham (banded sunflower moth); Cydia latiferreana Walsingham (filbertworm); C. pomonella Linnaeus (codling moth); Endopiza viteana Clemens (grape berry moth); Eupoecilia ambiguella Hubner (vine moth); Grapholita molesta Busck (oriental fruit moth); Lobesia botrana Denis & Schiffermuller (European grape vine moth); Platynota flavedana Clemens (variegated leafroller); P. stultana Walsingham (omnivorous leafroller); Spilonota ocellana Denis & Schiffermüller (eyespotted bud moth); and Suleima helianthana Riley (sunflower bud moth).

Additional Lepidoptera agronomic pests may include, but are not limited to, Alsophila pometaria Harris (fall cankerworm); Anarsia lineatella Zeller (peach twig borer); Anisota senatoria J. E. Smith (orange striped oakworm); Antheraea pernyi Guérin-Méneville (Chinese Oak Silkmoth); Bombyx mori Linnaeus (Silkworm); Bucculatrix thurberiella Busck (cotton leaf perforator); Collas eurytheme Boisduval (alfalfa caterpillar); Datana integerrima Grote & Robinson (walnut caterpillar); Dendrolimus sibiricus Tschetwerikov (Siberian silk moth), Ennomos subsignaria Hubner (elm spanworm); Erannis tiliaria Harris (linden looper); Erechthias flavistriata Walsingham (sugarcane bud moth); Euproctis chrysorrhoea Linnaeus (browntail moth); Harrisina americana Guérin-Méneville (grapeleaf skeletonizer); Heliothis subflexa Guenee; Hemileuca oliviae Cockrell (range caterpillar); Hyphantria cunea Drury (fall webworm); Keiferia lycopersicella Walsingham (tomato pinworm); Lambdina fiscellaria fiscellaria Hulst (Eastern hemlock looper); L. fiscellaria lugubrosa Hulst (Western hemlock looper); Leucoma salicis Linnaeus (satin moth); Lymantria dispar Linnaeus (gypsy moth); Malacosoma spp.; Manduca quinquemaculata Haworth (five spotted hawk moth, tomato hornworm); M. sexta Haworth (tomato hornworm, tobacco hornworm); Operophtera brumata Linnaeus (winter moth); Orgyia spp.; Paleacrita vernata Peck (spring cankerworm); Papilio cresphontes Cramer (giant swallowtail, orange dog); Phryganidia californica Packard (California oakworm); Phyllocnistis citrella Stainton (citrus leafminer); Phyllonorycter blancardella Fabricius (spotted tentiform leafminer); Pieris brassicae Linnaeus (large white butterfly); P. rapae Linnaeus (small white butterfly); P. napi Linnaeus (green veined white butterfly); Platyptilia carduidactyla Riley (artichoke plume moth); Plutella xylostella Linnaeus (diamondback moth); Pectinophora gossypiella Saunders (pink bollworm); Pontia protodice Boisduval & Leconte (Southern cabbageworm); Sabulodes aegrotata Guenee (omnivorous looper); Schizura concinna J. E. Smith (red humped caterpillar); Sitotroga cerealella Olivier (Angoumois grain moth); Thaumetopoea pityocampa Schiffermuller (pine processionary caterpillar); Tineola bisselliella Hummel (webbing clothesmoth); Tuta absoluta Meyrick (tomato leafminer) and Yponomeuta padella Linnaeus (ermine moth).

In certain embodiments, insect pests may include those of the order Hemiptera including, but not limited to, the following families: Adelgidae, Aleyrodidae, Aphididae, Asterolecaniidae, Cercopidae, Cicadellidae, Cicadidae, Cixiidae, Coccidae, Coreidae, Dactylopiidae, Delphacidae, Diaspididae, Eriococcidae, Flatidae, Fulgoridae, lssidae, Lygaeidae, Margarodidae, Membracidae, Miridae, Ortheziidae, Pentatomidae, Phoenicococcidae, Phylloxeridae, Pseudococcidae, Psyllidae, Pyrrhocoridae and Tingidae.

Non-limiting examples of agronomically important insect pests from the order Hemiptera include: Acrosternum hilare Say (green stink bug); Acyrthisiphon pisum Harris (pea aphid); Adelges spp. (adelgids); Adelphocoris rapidus Say (rapid plant bug); Anasa tristis De Geer (squash bug); Aphis craccivora Koch (cowpea aphid); A. fabae Scopoli (black bean aphid); A. gossypii Glover (cotton aphid, melon aphid); A. maidiradicis Forbes (corn root aphid); A. pomi De Geer (apple aphid); A. spiraecola Patch (spirea aphid); Aulacaspis tegalensis Zehntner (sugarcane scale); Aulacorthum solani Kaltenbach (foxglove aphid); Bemisia tabaci Gennadius (tobacco whitefly, sweetpotato whitefly); B. argentifolii Bellows & Perring (silverleaf whitefly); Blissus leucopterus leucopterus Say (chinch bug); Blostomatidae spp.; Brevicoryne brassicae Linnaeus (cabbage aphid); Cacopsylla pyricola Foerster (pear psylla); Calocoris norvegicus Gmelin (potato capsid bug); Chaetosiphon fragaefolii Cockerell (strawberry aphid); Cimicidae spp.; Coreidae spp.; Corythuca gossypii Fabricius (cotton lace bug); Cyrtopeltis modesta Distant (tomato bug); C. notatus Distant (suckfly); Deois flavopicta St\dot{a}l (spittlebug); Dialeurodes citri Ashmead (citrus whitefly); Diaphnocoris chlorionis Say (honeylocust plant bug); Diuraphis noxia Kurdjumov/Mordvilko (Russian wheat aphid); Duplachionaspis divergens Green (armored scale); Dysaphis plantaginea Paaserini (rosy apple aphid); Dysdercus suturellus Herrich-Schaffer (cotton stainer); Dysmicoccus boninsis Kuwana (gray sugarcane mealybug); Empoasca fabae Harris (potato leafhopper); Eriosoma lanigerum Hausmann (woolly apple aphid); Erythroneoura spp. (grape leafhoppers); Eumetopina flavipes Muir (Island sugarcane planthopper); Eurygaster spp.; Euschistus servus Say (brown stink bug); E. variolarius Palisot de Beauvois (one-spotted stink bug); Graptostethus spp. (complex of seed bugs); and Hyalopterus pruni Geoffroy (mealy plum aphid); Icerya purchasi Maskell (cottony cushion scale); Labopidicola allii Knight (onion plant bug); Laodelphax striatellus Fallen (smaller brown planthopper); Leptoglossus corculus Say (leaf-footed pine seed bug); Leptodictya tabida Herrich-Schaeffer (sugarcane lace bug); Lipaphis erysimi Kaltenbach (turnip aphid); Lygocoris pabulinus Linnaeus (common green capsid); Lygus lineolaris Palisot de Beauvois (tarnished plant bug); L. Hesperus Knight (Western tarnished plant bug); L. pratensis Linnaeus (common meadow bug); L. rugulipennis Poppius (European tarnished plant bug); Macrosiphum euphorbiae Thomas (potato aphid); Macrosteles quadrilineatus Forbes (aster leafhopper); Magicicada septendecim Linnaeus (periodical cicada); Mahanarva fimbriolata St\dot{a}l (sugarcane spittlebug); Melanaphis sacchari Zehntner (sugarcane aphid); Melanaspis glomerata Green (black scale); Metopolophium dirhodum Walker (rose grain aphid); Myzus persicae Sulzer (peach-potato aphid, green peach aphid); Nasonovia ribisnigri Mosley (lettuce aphid); Nephotettix cinticeps Uhler (green leafhopper); N. nigropictus St\dot{a}l (rice leafhopper); Nezara viridula Linnaeus (southern green stink bug); Nilaparvata lugens St\dot{a}l (brown planthopper); Nysius ericae Schilling (false chinch bug); Nysius raphanus Howard (false chinch bug); Oebalus pugnax Fabricius (rice stink bug); Oncopeltus fasciatus Dallas (large milkweed bug); Orthops campestris Linnaeus; Pemphigus spp. (root aphids and gall aphids); Peregrinus maidis Ashmead (corn planthopper); Perkinsiella saccharicida Kirkaldy (sugarcane delphacid); Phylloxera devastatrix Pergande (pecan phylloxera); Planococcus citri Risso (citrus mealybug); Plesiocoris rugicolfs Fallen (apple capsid); Poecilocapsus lineatus Fabricius (four-lined plant bug); Pseudatomoscelis seriatus Reuter (cotton fleahopper); Pseudococcus spp. (other mealybug complex); Pulvinaria elongata Newstead (cottony grass scale); Pyrilla perpusilla Walker (sugarcane leafhopper); Pyrrhocoridae spp.; Quadraspidiotus perniciosus Comstock (San Jose scale); Reduviidae spp.; Rhopalosiphum maidis Fitch (corn leaf aphid); R. padi Linnaeus (bird cherry-oat aphid); Saccharicoccus sacchari Cockerell (pink sugarcane mealybug); Schizaphis graminum Rondani (greenbug); Sipha flava Forbes (yellow sugarcane aphid); Sitobion avenae Fabricius (English grain aphid); Sogatella furcifera Horvath (white-backed planthopper); Sogatodes oryzicola Muir (rice delphacid); Spanagonicus albofasciatus Reuter (whitemarked fleahopper); Therioaphis maculata Buckton (spotted alfalfa aphid); Tinidae spp.; Toxoptera aurantii Boyer de Fonscolombe (black citrus aphid); and T. citricida Kirkaldy (brown citrus aphid); Trialeurodes abutiloneus (bandedwinged whitefly) and T. vaporariorum Westwood (greenhouse whitefly); Trioza diospyri Ashmead (persimmon psylla); and Typhlocyba pomaria McAtee (white apple leafhopper).

In other embodiments, insect pests may also include adults and larvae of the order Acari (mites) including, but not limited to, Aceria tosichella Keifer (wheat curl mite); Panonychus ulmi Koch (European red mite); Petrobia latens Muller (brown wheat mite); Steneotarsonemus bancrofti Michael (sugarcane stalk mite); spider mites and red mites in the family Tetranychidae, Oligonychus grypus Baker & Pritchard, O. indicus Hirst (sugarcane leaf mite), O. pratensis Banks (Banks grass mite), O. stickneyi McGregor (sugarcane spider mite); Tetranychus urticae Koch (two spotted spider mite); T. mcdanieli McGregor (McDaniel mite); T. cinnabarinus Boisduval (carmine spider mite); T. turkestani Ugarov & Nikolski (strawberry spider mite), flat mites in the family Tenuipalpidae, Brevipalpus lewisi McGregor (citrus flat mite); rust and bud mites in the family Eriophyidae and other foliar feeding mites and mites important in human and animal health, i.e. dust mites in the family Epidermoptidae, follicle mites in the family Demodicidae, grain mites in the family Glycyphagidae, ticks in the order Ixodidae. Ixodes scapularis Say (deer tick); I. holocyclus Neumann (Australian paralysis tick); Dermacentor variabilis Say (American dog tick); Amblyomma americanum Linnaeus (lone star tick); and scab and itch mites in the families Psoroptidae, Pyemotidae, and Sarcoptidae.

In addition, insect pests may also include those of the order Thysanura, such as Lepisma saccharina Linnaeus (silverfish) and Thermobia domestica Packard (firebrat).

Insect pests may also include those of the order Isoptera, including those of the termitidae family, such as, but not limited to, Cylindrotermes nordenskioeldi Holmgren and Pseudacanthotermes militaris Hagen (sugarcane termite).

Insect pests may also include those of the order Thysanoptera, including but not limited to thrips, such as Stenchaetothrips minutus van Deventer (sugarcane thrips).

In other embodiments, arthropod pests may include: spiders in the order Araneae such as Loxosceles reclusa Gertsch & Mulaik (brown recluse spider); and the Latrodectus mactans Fabricius (black widow spider); and centipedes in the order Scutigeromorpha such as Scutigera coleoptrata Linnaeus (house centipede).

As used herein, “pesticidal activity,” “insecticidal,” “pesticidal,” or “insecticidal activity” means that the proteins, polypeptides, or toxins of the present disclosure, including proteins that have homology to such proteins, polypeptides, or toxins, are able to induce the stunting (sub-lethal effect) and/or killing (lethal effect) of insect pathogens or pests, including but not limited to, members of the Lepidoptera, Diptera, Hemiptera, and Coleoptera orders or the Nematoda phylum.

In certain embodiments described herein, the compositions, plants, cells, and methods may further comprise at least one additional pesticidal protein as a pesticidal stacking partner to help, for example, in reducing the likelihood of resistance development or in expanding the spectrum of insect inhibition. These additional pesticidal proteins can be isolated from organisms including, for example, Bacillus sp., Pseudomonas sp., Photorhabdus sp., Xenorhabdus sp., Clostridium bifermentans, and Paenibacillus popilliae. In some embodiments, transgenic or modified plants expressing insecticidal proteins as described herein may also be crossed by breeding with transgenic events expressing other insecticidal proteins and/or expressing other transgenic traits such as other insect control traits, herbicide tolerance genes, genes conferring yield or stress tolerance traits, and the like, or such traits can be combined in a single vector so that the traits are all linked.

Additional pesticidal proteins may include, but are not limited to: insecticidal proteins from Pseudomonas sp. such as PSEEN3174 (Monalysin; (2011) PLoS Pathogens 7: 1-13); from Pseudomonas protegens strain CHAO and Pf-5 (previously fluorescens) (Pechy-Tarr, (2008) Environmental Microbiology 10:2368-2386; GenBank Accession No. EU400157); from Pseudomonas taiwanensis (Liu, et al., (2010) J. Agric. Food Chem., 58: 12343-12349) and from Pseudomonas pseudoalcaligenes (Zhang, et al., (2009) Annals of Microbiology 59:45-50 and Li, et al., (2007) Plant Cell Tiss. Organ Cult. 89: 159-168); insecticidal proteins from Photorhabdus sp. and Xenorhabdus sp. (Hinchliffe, et al., (2010) The Open Toxicology Journal, 3: 101-1 18 and Morgan, et al., (2001) Applied and Envir. Micro. 67:2062-2069); U.S. Pat. Nos. 6,048,838, and 6,379,946; a PIP-1 polypeptide of U.S. Pat. No. 9,688,730; an AflP-1A and/or AflP-1 B polypeptide of U.S. Pat. No. 9,475,847; a PIP-47 polypeptide of U.S. Pub. Number US20160186204; an IPD045 polypeptide, an IPD064 polypeptide, an IPD074 polypeptide, an IPD075 polypeptide, and an IPD077 polypeptide of PCT Pub. No. WO 2016/114973; an IPD080 polypeptide of PCT Serial Number PCT/US17/56517; an IPD078 polypeptide, an IPD084 polypeptide, an IPD085 polypeptide, an IPD086 polypeptide, an IPD087 polypeptide, an IPD088 polypeptide, and an IPD089 polypeptide of Serial Number PCT/US17/54160; PIP-72 polypeptide of U.S. Pat. Pub. No. US20160366891; a PtlP-50 polypeptide and a PtlP-65 polypeptide of U.S. Pub. Number US20170166921; an IPD098 polypeptide, an IPD059 polypeptide, an IPD108 polypeptide, an IPD109 polypeptide of U.S. Ser. No. 62/521,084; an IPD 110 polypeptide, an IPD 113 polypeptide, a PtlP-83 polypeptide of U.S. Pub. Number US20160347799; a PtlP-96 polypeptide of U.S. Pub. Number US20170233440; an IPD079 polypeptide of PCT Pub. No. WO2017/23486; an IPD082 polypeptide of PCT Pub. No. WO 2017/105987, an IPD090 polypeptide of Serial Number PCT/US 17/30602, an IPD093 polypeptide of U.S. Ser. No. 62/434,020; an IPD103 polypeptide of Serial Number PCT/US 17/39376; an IPD101 polypeptide of U.S. Ser. No. 62/438,179; an IPD121 polypeptide of U.S. Serial Number U.S. 62/508,514; and delta (d)-endotoxins including, but not limited to a Cry1, Cry2, Cry3, Cry4, Cry5, Cry6, Cry7, Cry8, Cry9, Cry10, Cry11, Cry12, Cry13, Cry14, Cry15, Cry16, Cry17, Cry18, Cry19, Cry20, Cry21, Cry22, Cry23, Cry24, Cry25, Cry26, Cry27, Cry28, Cry29, Cry30, Cry31, Cry32, Cry33, Cry34, Cry35,Cry36, Cry37, Cry38, Cry39, Cry40, Cry41, Cry42, Cry43, Cry44, Cry45, Cry46, Cry47, Cry49, Cry50, Cry51, Cry52, Cry53, Cry54, Cry55, Cry56, Cry57, Cry58, Cry59, Cry60, Cry61, Cry62, Cry63, Cry64, Cry65, Cry66, Cry67, Cry68, Cry69, Cry70, Cry71, and Cry72 classes of d-endotoxin polypeptides and the B. thuringiensis cytolytic cyt1 and cyt2 genes. Members of these classes of B. thuringiensis insecticidal proteins (see Crickmore et al., “Bacillus thuringiensis toxin nomenclature” (2011), at bpprc.org).

Examples of d-endotoxins also include, but are not limited to: Cry1A proteins of U.S. Pat. Nos. 5,880,275, 7,858,849, and 8,878,007; a Cry1Ac mutant of U.S. Pat. No. 9,512,187; a DIG-3 or DIG-1 1 toxin (N-terminal deletion of a-helix 1 and/or a-helix 2 variants of cry proteins such as Cry1A, Cry3A) of U.S. Pat. No. 8,304,604, 8,304,605 and 8,476,226; Cry1B of U.S. patent application Ser. No. 10/525,318, U.S. Pat. App. Pub. No. US20160194364, and U.S. Pat. Nos. 9,404,121 and 8,772,577; Cry1B variants of PCT Pub. No. WO2016/61197 and Serial Number PCT/US17/27160; Cry1C of U.S. Pat. No. 6,033,874; Cry1D protein of US20170233759; a Cry1E protein of PCT Serial Number PCT/US17/53178; a Cry1F protein of U.S. Pat. Nos. 5,188,960 and 6,218,188; Cry1A/F chimeras of U.S. Pat. No. 7,070,982; 6,962,705 and 6,713,063; a Cry1L protein of PCT Pub. No. WO 2017/0233759; an engineered Cry1G as set forth in WO2018111553A1; a CryU variant of U.S. Pub. US20170240603; a Cry2 protein such as Cry2Ab protein of U.S. Pat. No. 7,064,249 and Cry2A.127 protein of U.S. Pat. No. 7,208,474; a Cry3A protein including but not limited to an engineered hybrid insecticidal protein (eHIP) created by fusing unique combinations of variable regions and conserved blocks of at least two different Cry proteins (U.S. Pat. App. Pub. No. 2010/0017914); a Cry4 protein; a Cry5 protein; a Cry6 protein; Cry8 proteins of U.S. Pat. Nos. 7,329,736, 7,449,552, 7,803,943, 7,476,781, 7,105,332, 7,339,092, 7,378,499, 7,462,760, and 9,593,345; a Cry9 protein such as such as members of the Cry9A, Cry9B, Cry9C, Cry9D, Cry9E and Cry9F families including the Cry9 protein of U.S. Pat. Nos. 9,000,261 and 8,802,933, and WO 2017/132188; a Cry15 protein of Naimov, et al., (2008) Applied and Environmental Microbiology, 74:7145-7151; a Cry14 protein of U.S. Pat. No. 8,933,299; a Cry22, a Cry34Abl protein of U.S. Pat. Nos. 6,127,180, 6,624,145, and 6,340,593; a truncated Cry34 protein of U.S. Pat. No. 8,816,157; a CryET33 and cryET34 protein of U.S. Pat. Nos. 6,248,535, 6,326,351, 6,399,330, 6,949,626, 7,385,107 and 7,504,229; a CryET33 and CryET34 homologs of U.S. Pat. Pub. No. 2006/0191034, 2012/0278954, and PCT Pub. No. WO 2012/139004; a Cry35Abl protein of U.S. Pat. Nos. 6,083,499, 6,548,291 and 6,340,593; a Cry46 protein of U.S. Pat. No. 9,403,881, a Cry51 protein, a Cry binary toxin; a TIC901 or related toxin; TIC807 of U.S. Pat. App. Pub. No. 2008/0295207; TIC853 of U.S. Pat. No. 8,513,493; ET29, ET37, TIC809, TIC810, TIC812, TIC127, TIC128 of PCT US2006/033867; engineered Hemipteran toxic proteins of U.S. Pat. App. Pub. No. US20160150795, AXMI-027, AXMI-036, and AXMI-038 of U.S. Pat. No. 8,236,757; AXMI-031, AXMI-039, AXMI-040, AXMI-049 of U.S. Pat. No. 7,923,602; AXMI-018, AXMI-020 and AXMI-021 of WO 2006/083891; AXMI-010 of WO 2005/038032; AXMI-003 of WO 2005/021585; AXMI-008 of U.S. Pat. App. Pub. No. 2004/0250311; AXMI-006 of U.S. Pat. App. Pub. No. 2004/0216186; AXMI-007 of U.S. Pat. App. Pub. No. 2004/0210965; AXMI-009 of U.S. Pat. Application Number 2004/0210964; AXMI-014 of U.S. Pat. App. Pub. No. 2004/0197917; AXMI-004 of U.S. Pat. App. Pub. No. 2004/0197916; AXMI-028 and AXMI-029 of WO 2006/119457; AXMI-007, AXMI-008, AXMI-0080rf2, AXMI-009, AXMI-014 and AXMI-004 of WO 2004/074462; AXMI-150 of U.S. Pat. No. 8,084,416; AXMI-205 of U.S. Pat. App. Pub. No. 2011/0023184; AXMI-011, AXMI-012, AXMI-013, AXMI-015, AXMI-019, AXMI-044, AXMI-037, AXMI-043, AXMI-033, AXMI-034, AXMI-022, AXMI-023, AXMI-041, AXMI-063 and AXMI-064 of U.S. Pat. App. Pub. No. 2011/0263488; AXMI046, AXMI048, AXMI050, AXMI051, AXMI052, AXMI053, AXMI054, AXMI055, AXMI056, AXMI057, AXMI058, AXMI059, AXMI060, AXMI061, AXMI067, AXMI069, AXMI071, AXMI072, AXMI073, AXMI074, AXMI075, AXMI087, AXMI088, AXMI093, AXMI070, AXMI080, AXMI081, AXMI082, AXMI091, AXMI092, AXMI096, AXMI097, AXMI098, AXMI099, AXMI 100, AXMI 101, AXMI 102, AXMI 103, AXMI 104, AXMI 107, AXMI 108, AXMI109, AXMI110, AXMI111, AXMI112, AXMI114, AXMI116, AXMI117, AXMI118, AXMI119, AXMI120, AXMI121, AXMI122, AXMI123, AXMI124, AXMI125, AXMI126, AXMI127, AXMI129, AXMI151, AXMI161, AXMI164, AXMI183, AXMI132, AXMI137, AXMI138 of U.S. Pat. Nos. 8,461,421 and 8,461,422; AXMI-R1 and related proteins of U.S. Pat. App. Pub. No. 2010/0197592; AXMI221Z, AXMI222z, AXMI223z, AXMI224z and AXMI225Z of WO 2011/103248; AXMI218, AXMI219, AXMI220, AXMI226, AXMI227, AXMI228, AXMI229, AXMI230 and AXMI231 of WO 2011/103247; AXMI-115, AXMI-113, AXMI-005, AXMI-163 and AXMI-184 of U.S. Pat. No. 8,334,431; AXMI-001, AXMI-002, AXMI-030, AXMI-035 and AXMI-045 of U.S. Pat. App. Pub. No. 2010/0298211; AXMI-066 and AXMI-076 of U.S. Pat. App. Pub. No. 2009/0144852; AXMI128, AXMI130, AXMI131, AXMI133, AXMI140, AXMI141, AXMI142, AXMI143, AXMI144, AXMI146, AXMI148, AXMI149, AXMI152, AXMI153, AXMI154, AXMI155, AXMI156, AXMI157, AXMI158, AXMI162, AXMI165, AXMI166, AXMI167, AXMI168, AXMI169, AXMI170, AXMI171, AXMI172, AXMI173, AXMI174, AXMI175, AXMI176, AXMI177, AXMI178, AXMI179, AXMI180, AXMI181, AXMI182, AXMI185, AXMI186, AXMI187, AXMI188, AXMI189 of U.S. Pat. No. 8,318,900; AXMI079, AXMI080, AXMI081, AXMI082, AXMI091, AXMI092, AXMI096, AXMI097, AXMI098, AXMI099, AXMI100, AXMI101, AXMI102, AXMI103, AXMI104, AXMI107, AXMI108, AXMI109, AXMI110, dsAXMI111, AXMI1 12, AXMI1 14, AXMI116, AXMI117, AXMI118, AXMI119, AXMI120, AXMI121, AXMI122, AXMI123, AXMI124, AXMI1257, AXMI1268, AXMI127, AXMI129, AXMI164, AXMI151, AXMI161, AXMI183, AXMI132, AXMI138, AXMI137 of U.S. Pat. No. 8,461,421; AXMI192 of U.S. Pat. No. 8,461,415; AXMI281 of U.S. Pat. App. Pub. No. US20160177332; AXMI422 of U.S. Pat. No. 8,252,872; cry proteins such as Cry1A and Cry3A having modified proteolytic sites of U.S. Pat. No. 8,319,019; a Cry1Ac, Cry2Aa and Cry1Ca toxin protein from Bacillus thuringiensis strain VBTS 2528 of U.S. Pat. App. Pub. No. 2011/0064710; and the Cry proteins MP032, MP049, MP051, MP066, MP068, MP070, MP091S, MP109S, MP114, MP121, MP134S, MP183S, MP185S, MP186S, MP195S, MP197S, MP208S, MP209S, MP212S, MP214S, MP217S, MP222S, MP234S, MP235S, MP237S, MP242S, MP243, MP248, MP249S, MP251M, MP252S, MP253, MP259S, MP287S, MP288S, MP295S, MP296S, MP297S, MP300S, MP304S, MP306S, MP310S, MP312S, MP314S, MP319S, MP325S, MP326S, MP327S, MP328S, MP334S, MP337S, MP342S, MP349S, MP356S, MP359S, MP360S, MP437S, MP451S, MP452S, MP466S, MP468S, MP476S, MP482S, MP522S, MP529S, MP548S, MP552S, MP562S, MP564S, MP566S, MP567S, MP569S, MP573S, MP574S, MP575S, MP581 S, MP590, MP594S, MP596S, MP597, MP599S, MP600S, MP601 S, MP602S, MP604S, MP626S, MP629S, MP630S, MP631S, MP632S, MP633S, MP634S, MP635S, MP639S, MP640S, MP644S, MP649S, MP651S, MP652S, MP653S, MP661 S, MP666S, MP672S, MP696S, MP704S, MP724S, MP729S, MP739S, MP755S, MP773S, MP799S, MP800S, MP801S, MP802S, MP803S, MP805S, MP809S, MP815S, MP828S, MP831 S, MP844S, MP852, MP865S, MP879S, MP887S, MP891S, MP896S, MP898S, MP935S, MP968, MP989, MP993, MP997, MP1049, MP1066, MP1067, MP1080, MP1081, MP1200, MP1206, MP1233, and MP1311 of U.S. Ser. No. 62/607,372.

Other exemplary additional pesticidal proteins for the control of Lepidopteran pests may include an insect inhibitory protein such as, but not limited to, Cry1A (U.S. Pat. No. 5,880,275), Cry1Ab, Cry1Ac, Cry1A.105, Cry1Ae, Cry1B (U.S. Pat. Pub. Ser. No. 10/525,318), Cry1C (U.S. Pat. No. 6,033,874), Cry1D, Cry1E, Cry1F, and Cry1A/F chimeras (U.S. Pat. Nos. 7,070,982; 6,962,705; and 6,713,063), Cry1G, Cry1H, Cry1I, Cry1J, Cry1K, Cry1L, Cry2A, Cry2Ab (U.S. Pat. No. 7,064,249), Cry2Ae, Cry4B, Cry6, Cry7, Cry8, Cry9, Cry15, Cry43A, Cry43B, Cry51Aal, ET66, TIC400, TIC800, TIC834, TIC1415, Vip3A, VIP3Ab, VIP3B, AXMI-001, AXMI-002, AXMI-030, AXMI-035, AXMI-045 (U.S. Pat. Pub. 2013-0117884 A1), AXMI-52, AXMI-58, AXMI-88, AXMI-97, AXMI-102, AXMI-112, AXMI-117, AXMI-100 (U.S. Pat. Pub. 2013-0310543 A1), AXMI-115, AXMI-113, AXMI-005 (U.S. Pat. Pub. 2013-0104259 A1), AXMI-134 (U.S. Pat. Pub. 2013-0167264 A1), AXMI-150 (U.S. Pat. Pub. 2010-0160231 A1), AXMI-184 (U.S. Pat. Pub. 2010-0004176 A1), AXMI-196, AXMI-204, AXMI-207, AXMI-209 (U.S. Pat. Pub. 2011-0030096 A1), AXMI-218, AXMI-220 (U.S. Pat. Pub. 2014-0245491 A1), AXMI-221z, AXMI-222z, AXMI-223z, AXMI-224z, AXMI-225z (U.S. Pat. Pub. 2014-0196175 A1), AXMI-238 (U.S. Pat. Pub. 2014-0033363 A1), AXMI-270 (U.S. Pat. Pub. 2014-0223598 A1), AXMI-345 (U.S. Pat. Pub. 2014-0373195 A1), DIG-3 (U.S. Pat. Pub. 2013-0219570 A1), DIG-5 (U.S. Pat. Pub. 2010-0317569 A1), DIG-11 (U.S. Pat. Pub. 2010-0319093 A1), AfIP-1A and derivatives thereof (U.S. Pat. Pub. 2014-0033361 A1), AfIP-1B and derivatives thereof (U.S. Pat. Pub. 2014-0033361 A1), PIP-1APIP-1B (U.S. Pat. Pub. 2014-0007292 A1), PSEEN3174 (U.S. Pat. Pub. 2014-0007292 A1), AECFG-592740 (U.S. Pat. Pub. 2014-0007292 A1), Pput 1063 (U.S. Pat. Pub. 2014-0007292 A1), Pput_1064 (U.S. Pat. Pub. 2014-0007292 A1), GS-135 and derivatives thereof (U.S. Pat. Pub. 2012-0233726 A1), GS153 and derivatives thereof (U.S. Pat. Pub. 2012-0192310 A1), GS154 and derivatives thereof (U.S. Pat. Pub. 2012-0192310 A1), GS155 and derivatives thereof (U.S. Pat. Pub. 2012-0192310 A1), SEQ ID NO: 2 and derivatives thereof as described in U.S. Pat. Pub. 2012-0167259 A1, SEQ ID NO: 2 and derivatives thereof as described in U.S. Pat. Pub. 2012-0047606 A1, SEQ ID NO: 2 and derivatives thereof as described in U.S. Pat. Pub. 2011-0154536 A1, SEQ ID NO: 2 and derivatives thereof as described in U.S. Pat. Pub. 2011-0112013 A1, SEQ ID NO: 2 and 4 and derivatives thereof as described in U.S. Pat. Pub. 2010-0192256 A1, SEQ ID NO: 2 and derivatives thereof as described in U.S. Pat. Pub. 2010-0077507 A1, SEQ ID NO: 2 and derivatives thereof as described in U.S. Pat. Pub. 2010-0077508 A1, SEQ ID NO: 2 and derivatives thereof as described in U.S. Pat. Pub. 2009-0313721 A1, SEQ ID NO: 2 or 4 and derivatives thereof as described in U.S. Pat. Pub. 2010-0269221 A1, SEQ ID NO: 2 and derivatives thereof as described in U.S. Pat. No. 7,772,465 B2, CF161_0085 and derivatives thereof as described in WO2014/008054 A2, Lepidopteran toxic proteins and their derivatives as described in U.S. Pat. Pub. 2008-0172762 A1, 2011-0055968 A1, and 2012-0117690 A1; SEQ ID NO: 2 and derivatives thereof as described in U.S. Pat. No. 7,510,878 B2, SEQ ID NO: 2 and derivatives thereof as described in U.S. Pat. No. 7,812,129 B1; and the like.

In other embodiments, exemplary additional pesticidal proteins for the control of Coleopteran pests may include an insect inhibitory protein such as, but not limited to, Cry3Bb (U.S. Pat. No. 6,501,009), Cry1C variants, Cry3A variants, Cry3, Cry3B, Cry34/35, 5307, AXMI-134 (U.S. Pat. Pub. 2013-0167264 A1) AXMI-184 (U.S. Pat. Pub. 2010-0004176 A1), AXMI-205 (U.S. Pat. Pub. 2014-0298538 A1), AXMI-207 (U.S. Pat. Pub. 2013-0303440 A1), AXMI-218, AXMI-220 (U.S. Pat. Pub. 20140245491A1), AXMI-221z, AXMI-223z (U.S. Pat. Pub. 2014-0196175 A1), AXMI-279 (U.S. Pat. Pub. 2014-0223599 A1), AXMI-R1 and variants thereof (U.S. Pat. Pub. 2010-0197592 A1), TIC407, TIC417, TIC431, TIC807, TIC853, TIC901, TIC1201, TIC3131, DIG-10 (U.S. Pat. Pub. 2010-0319092 A1), eHIPs (U.S. Pat. Pub. 2010/0017914), IP3 and variants thereof (U.S. Pat. Pub. 2012-0210462 A1), and o-Hexatoxin-Hv1a (U.S. Pat. Pub. 2014-0366227 A1).

In other embodiments, exemplary additional pesticidal proteins for the control of Hemipteran pests may include Hemipteran-active proteins such as, but not limited to, TIC1415 (U.S. Pat. Pub. 2013-0097735 A1), TIC807 (U.S. Pat. No. 8,609,936), TIC834 (U.S. Pat. Pub. 2013-0269060 A1), AXMI-036 (U.S. Pat. Pub. 2010-0137216 A1), and AXMI-171 (U.S. Pat. Pub. 2013-0055469 A1).

Additional pesticidal proteins for the control of Coleopteran, Lepidopteran, and Hemipteran insect pests can be found on the Bacillus thuringiensis toxin nomenclature website (btnomenclature.info).

As used herein, “plant cell” or “plant cells” means a cell obtained from or found in seeds, suspension cultures, embryos, meristematic regions, callus tissue, leaves, roots, shoots, gametophytes, sporophytes, pollen, protoplasts, and microspores. Plant cell also includes modified cells, such as protoplasts, obtained from the aforementioned tissues, as well as plant cell tissue cultures from which plants can be regenerated, plant calli and plant clumps. As used herein, “plant part” or “plant parts” means organs such as embryos, pollen, ovules, seeds, flowers, kernels, ears, cobs, leaves, husks, stalks, stems, roots, root tips, anthers, silk and the like. As used herein, “plant” or “plants” means whole plants and their progeny. Progeny, variants, and mutants of the regenerated plants are also included, provided that they comprise the introduced nucleic acid molecule as described herein.

“Promoter” as used herein means a synthetic or naturally derived molecule which is capable of conferring, activating, or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organelle in which expression occurs or, with respect to the developmental stage at which expression occurs, or in response to external stimuli such as physiological stresses, pathogens or pests, pesticides, metal ions, or inducing agents. Representative examples of promoters include the promoter of the 35S gene from the cauliflower mosaic virus, the promoter from the cassava vein mosaic virus, the promoter of the rice actin1 gene, the promoter of the subterranean clover virus gene 4, the promoter region of the ubiquitin 4 gene, and the promoter region of the maize polyubiquitin 1 gene. A “regulatory element” is a polynucleotide sequence that has an effect upon transcription of a gene. A regulatory element may include, but is not limited to, a promoter, enhancer, terminator, or other sequences that affect transcription of a gene.

The term “recombinant” when used with reference to, for example, a cell, nucleic acid, protein, or vector, indicates that the cell, nucleic acid, protein, or vector, has been modified by the introduction of a heterologous nucleic acid or protein or the alteration of a native nucleic acid or protein, or that the cell is derived from a cell so modified. Thus, for example, recombinant cells express genes that are not found within the native (naturally occurring) form of the cell or express a second copy of a native gene that is otherwise normally or abnormally expressed, under expressed, or not expressed at all.

“Sample” or “test sample” as used herein can mean any sample in which the presence and/or level of a target is to be detected or determined or any sample comprising a vector as detailed herein. The sample may be a biological sample. Samples may include liquids, solutions, emulsions, or suspensions. Samples can be obtained by any means known in the art. The sample can be used directly as obtained from an organism or can be pre-treated, such as by filtration, distillation, extraction, concentration, centrifugation, inactivation of interfering components, addition of reagents, and the like, to modify the character of the sample in some manner as discussed herein or otherwise as is known in the art.

“Subject” and “organism” as used herein interchangeably refer to any plant, seed, plant part, or plant material including, but not limited to, a plant in need of the herein described compositions or methods. The plant may be, for example but not limited to, rice, barley, sorghum, soybean, cotton, maize, rapeseed, sugar cane, tobacco, sunflower, or wheat. The subject may be at any stage of development, such as, for example, seed, sprout, vegetative, budding, flowering, or ripening stages. The subject may be hermaphrodite or dioecious. In some embodiments, the subject may have a specific genetic marker. In some embodiments, the subject may be undergoing other forms of treatment.

“Substantially identical” can mean that a first and second amino acid or polynucleotide sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or greater amino acids or nucleotides, respectively.

As used herein, “introducing” means presenting to the plant cell, plant part, or plant, a nucleic acid molecule or construct in such a manner that it gains access to the interior of a cell of the plant. Methods of the present disclosure include introducing and expressing in a plant cell, plant part, or plant a nucleic acid sequence or construct as described herein. The methods described herein do not depend on the particular method for introducing the nucleic acid molecule or nucleic acid construct into the plant cell, plant part, or plant, only that it gains access to the interior of at least one cell of the plant or plant part. Methods of introducing nucleotide sequence, selecting transformation event, and regenerating whole plants, which may require routine modification in respect of a particular plant species, are known in the art. The methods may include, but are not limited to, stable transformation methods, transient transformation methods, virus-mediated methods, and sexual breeding. As such, the nucleic acid molecule or construct can be carried episomally or integrated into the genome of the host cell.

“Transformed plant cells” as used herein refer to plant cells that have been transformed that can be grown into plants by methods known in the art. These plants can then be grown, and either pollinated with the same transformed strain or different strains, where the resulting progeny have the desired phenotypic characteristic identified. Two or more generations can be grown to ensure that expression of the desired phenotypic characteristic is stably maintained and inherited, and then seeds harvested to ensure expression of the desired phenotypic characteristic has been achieved.

“Transformation event” means a product of organism or cell transformation with a heterologous DNA construct, the regeneration of a population of organisms resulting from the insertion of the recombinant DNA into the genome of the organism, and selection of a particular organism characterized by insertion of the gene construct into a particular genome location resulting in a transgenic cell of organism.

“Transformed organisms” or “transformed plants” refers to organisms or plants having integrated into their genome a nucleic acid molecule heterologous to the organisms or plants. All cells of the transformed organisms or plants may have a genetic construct integrated into their genome. A transformed plant may be a fertile plant and more particularly a plant which agronomic properties (yield, grain quality, drought tolerance, etc.) are not impaired compared to the same plant not transformed. In some embodiments, organisms or plants are transformed using agrobacterium-mediated transformation. Other suitable transformation methods may include, for example, particle bombardment or silicon carbide whiskers, CRISPR, TALENs, or other genome modification techniques. Genome modification techniques may alter the genome of a plant through insertion or other alteration of the plant genome. In some embodiments, a modified plant comprising a nucleic acid encoding a polypeptide as disclosed herein is contemplated.

In some embodiments, the disclosed polynucleotides encoding a polypeptide may be introduced into the genome of a plant using genome editing technologies, or previously introduced polynucleotides in the genome of a plant may be edited using genome editing technologies. For example, the disclosed polynucleotides can be introduced into a desired location in the genome of a plant through the use of double-stranded break technologies including, but not limited to, TALENs, meganucleases, zinc finger nucleases, CRISPR-Cas, and the like. The disclosed polynucleotides may be introduced into a desired location in a plant genome using a CRISPR-Cas system for the purpose of site-specific insertion. The desired location in a plant genome may be any desired target site for insertion, such as a genomic region optimized for breeding, or may be a target site located in a genomic region with an existing trait of interest. Existing traits of interest could be either an endogenous trait or a previously introduced trait.

In some embodiments, where the disclosed polynucleotide encoding the insecticidal polypeptide has previously been introduced into a genome, genome editing technologies may be used to alter or modify the introduced polynucleotide encoding the insecticidal polypeptide sequence. Alternatively, double-stranded break technologies can be used to add additional nucleotide sequences to the introduced polynucleotide. Additional sequences that may be added include additional expression elements, such as enhancer and promoter sequences. In another embodiment, genome editing technologies may be used to position additional nucleotide sequences encoding additional insecticidally-active proteins in close proximity to the disclosed polynucleotide encoding the insecticidal polypeptide disclosed herein within the genome of a plant, in order to generate molecular stacks of insecticidally-active proteins.

“Transgene” as used herein refers to a gene or genetic material containing a gene sequence that has been isolated from one organism and is introduced into a different organism. This non-native segment of DNA may retain the ability to produce RNA or protein in the transgenic organism, or it may alter the normal function of the transgenic organism's genetic code. The introduction of a transgene has the potential to change the phenotype of an organism.

“Treatment” or “treating” when referring to protection of a subject from a toxin, means suppressing, repressing, reversing, alleviating, ameliorating, or inhibiting the progress of damage or death due to exposure to a toxin, or completely eliminating a damage or death due to exposure to a toxin. A treatment may be either performed in an acute or chronic way. Preventing damage or death due to exposure to a toxin involves administering a composition of the present disclosure to a subject prior to exposure to a toxin. Suppressing damage or death due to exposure to a toxin involves administering a composition of the present disclosure to a subject exposure to a toxin but before the appearance of damage. Repressing or ameliorating damage or death due to exposure to a toxin involves administering a composition of the present disclosure to a subject after the appearance of damage. Treatment may be from the expression of a transgene or topical application of a polypeptide of the disclosure to a plant.

“Variant,” with respect to a nucleotide or polynucleotide, means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequence substantially identical thereto.

“Variant,” with respect to a peptide, polypeptide, or protein, means differing in amino acid sequence by the insertion, deletion, or conservative substitution of amino acids, but retaining at least one biological activity. Variant may also mean a protein with an amino acid sequence that is substantially identical to a referenced protein with an amino acid sequence that retains at least one biological activity. Representative examples of “biological activity” include the ability to be bound by a specific polypeptide or to promote a specific response such as resistance or activity. Biological activity can also mean pesticidal or insecticidal activity. Variant can mean a functional fragment thereof, including functional truncated fragments and variants. Variant can also mean multiple copies of a polypeptide. The multiple copies can be in tandem or separated by a linker. A conservative substitution of an amino acid, for example, replacing an amino acid with a different amino acid of similar properties (for example, hydrophilicity, degree and distribution of charged regions) is recognized in the art as typically involving a minor change. These minor changes may be identified, in part, by considering the hydropathic index of amino acids, as understood in the art. The hydropathic index of an amino acid is based on a consideration of its hydrophobicity and charge. It is known in the art that amino acids of similar hydropathic indexes may be substituted and still retain protein function. The hydrophilicity of amino acids may also be used to reveal substitutions that would result in proteins retaining biological function. A consideration of the hydrophilicity of amino acids in the context of a peptide permits calculation of the greatest local average hydrophilicity of that peptide. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

In some embodiments, variant pesticidal proteins may be engineered by methods known in the art such that their sequence differs from a natural (i.e., native) or “wild-type” sequence. Protein engineering methods may be used to achieve, for example, improved pesticidal activities against specific pests (i.e., optimization) or altered target spectrum. As disclosed herein, suitable engineering methods for the generation of variant pesticidal proteins may include, but are not limited to, domain swapping, DNA shuffling, saturation mutagenesis, site-directed mutagenesis, oligonucleotide-mediated mutagenesis, cassette mutagenesis, and error-prone PCR techniques.

Variant nucleotide sequences and proteins disclosed herein encompass sequences and proteins derived from a mutagenic or recombinogenic procedure such as DNA shuffling. With such a procedure, one or more different coding sequences can be manipulated to create an engineered pesticidal protein possessing one or more desired properties. In this manner, libraries of recombinant polynucleotides can be generated from a population of related sequence polynucleotides comprising sequence regions that have substantial sequence identity and can be homologously recombined in vitro or in vivo. For example, using this approach, full-length coding sequences, sequence motifs encoding a domain of interest, or any fragment of a nucleotide sequence may be shuffled between nucleotide sequences encoding the pesticidal proteins described herein and other known pesticidal nucleotide sequences to obtain a new gene coding for an engineered protein having an improved property of interest, such as an increased insecticidal activity. Properties of interest may include, but are not limited to, pesticidal activity per unit of pesticidal protein, protein stability, and non-toxicity to non-target species, particularly humans, livestock, and plants and microbes that express the disclosed pesticidal proteins. DNA shuffling methods may involve only nucleotide sequences disclosed herein or may additionally involve shuffling of other nucleotide sequences known in the art. Strategies for such shuffling methods are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458.

Domain swapping is another known engineering mechanism for generating variant pesticidal proteins. For example, domains may be swapped between different pesticidal polypeptides, resulting in hybrid or chimeric fusion protein toxins having altered insecticidal activity or target spectrum. Methods for generating recombinant engineered proteins and testing them for pesticidal activity are known in the art. See, for example, Naimov, et al., (2001) Appl. Environ. Microbiol. 67:5328-5330; de Maagd, et al., (1996) Appl. Environ. Microbiol. 62:1537-1543; Ge, et al., (1991) J. Biol. Chem. 266:17954-17958; Schnepf, et al., (1990) J Biol. Chem. 265:20923-20930; and Rang, et al., 91999) Appl. Environ. Microbiol. 65:2918-2925.

Alternatively, variant nucleic acid sequences can be made by introducing mutations randomly along all or part of a nucleotide coding sequence, such as by saturation mutagenesis, and the resultant mutants can be screened for the ability to confer pesticidal activity to identify mutants that retain activity or have improved activity. Following mutagenesis, the encoded pesticidal protein can be expressed recombinantly, and the activity of the variant protein can be determined using standard assay techniques known in the art.

A protein of the present disclosure may be engineered to produce a different physical property, such as increased resistance or insecticidal activity to insects, altered insecticidal or resistance spectrum, or reduced plant phytotoxicity. An engineered protein may be a variant, mutant, fragment, or chimeric protein from a starting polypeptide sequence.

“Vector” as used herein means a nucleic acid sequence containing an origin of replication. A vector may be a bacterial plasmid, viral vector, bacteriophage, bacterial artificial chromosome, plant expression vector, animal expression vector, archaeal vector, or yeast artificial chromosome. A vector may be a DNA or RNA vector. A vector may be a self-replicating extrachromosomal vector, and may be a DNA plasmid. For example, the vector may encode a pesticidal protein.

Provided herein are nucleic acid molecules. A nucleic acid molecule may include a pesticidal gene polynucleotide such as that encoding any one of SEQ ID NOs: 1-56, a selectable marker gene to allow transgenic plant selection, and/or a visual reporter marker such as GFP. The nucleic acid molecule may also comprise a nucleic acid that encodes a fusion protein.

Nucleic acid molecules described herein may include, for example, polynucleotides such as vectors and plasmids. The vector may be an expression vector or system to produce protein by routine techniques and readily available starting materials. The polynucleotide may be recombinant. The polynucleotide may comprise regulatory elements for gene expression of the coding sequences of the nucleic acid. Coding sequences in the polynucleotide may be optimized for stability and high levels of expression. Regulatory elements may include a promoter, an enhancer, an initiation codon, a stop codon, and/or a polyadenylation signal.

In one aspect, the polynucleotide may encode a polypeptide having at least 70% sequence identity to any one of SEQ ID NOs: 1-56, and having pesticidal activity; the nucleic acid molecule may encode a polypeptide having at least 75% sequence identity to any one of SEQ ID NOs: 1-56, and having pesticidal activity; the nucleic acid molecule may encode a polypeptide having at least 80% sequence identity to any one of SEQ ID NOs: 1-56, and having pesticidal activity; the nucleic acid molecule may encode a polypeptide having at least 85% sequence identity to any one of SEQ ID NOs: 1-56, and having pesticidal activity; the nucleic acid molecule may encode a polypeptide having at least 90% sequence identity to any one of SEQ ID NOs: 1-56, and having pesticidal activity; the nucleic acid molecule may encode a polypeptide having at least 91% sequence identity to any one of SEQ ID NOs: 1-56, and having pesticidal activity; the nucleic acid molecule may encode a polypeptide having at least 92% sequence identity to any one of SEQ ID NOs: 1-56, and having pesticidal activity; the nucleic acid molecule may encode a polypeptide having at least 93% sequence identity to any one of SEQ ID NOs: 1-56, and having pesticidal activity; the nucleic acid molecule may encode a polypeptide having at least 94% sequence identity to any one of SEQ ID NOs: 1-56, and having pesticidal activity; the nucleic acid molecule may encode a polypeptide having at least 95% sequence identity to any one of SEQ ID NOs: 1-56, and having pesticidal activity; the nucleic acid molecule may encode a polypeptide having at least 96% sequence identity to any one of SEQ ID NOs: 1-56, and having pesticidal activity; the nucleic acid molecule may encode a polypeptide having at least 97% sequence identity to any one of SEQ ID NOs: 1-56, and having pesticidal activity; the nucleic acid molecule may encode a polypeptide having at least 98% sequence identity to any one of SEQ ID NOs: 1-56, and having pesticidal activity; the nucleic acid molecule may encode a polypeptide having at least 99% sequence identity to any one of SEQ ID NOs: 1-56, and having pesticidal activity; the nucleic acid molecule may encode a polypeptide having at least 99.2% sequence identity to any one of SEQ ID NOs: 1-56, and having pesticidal activity; the nucleic acid molecule may encode a polypeptide having at least 99.5% sequence identity to any one of SEQ ID NOs: 1-56, and having pesticidal activity; the nucleic acid molecule may encode a polypeptide having at least 99.8% sequence identity to any one of SEQ ID NOs: 1-56, and having pesticidal activity; the nucleic acid molecule may encode a polypeptide having at least 99.9% sequence identity to any one of SEQ ID NOs: 1-56, and having pesticidal activity; or, the nucleic acid molecule may encode a polypeptide having the amino acid sequence of any one of SEQ ID NOs: 1-56, and having pesticidal activity.

In one embodiment, the present disclosure is directed to an isolated polynucleotide encoding a polypeptide amino acid sequence having at least 80% or at least 95% sequence identity to any one of SEQ ID NOs: 1-56, and having pesticidal activity. The pesticidal polypeptides and polynucleotides encoding the pesticidal polypeptides of the present disclosure are particularly useful in agricultural crops for controlling and killing pests.

In one aspect, the present disclosure is directed to a method for producing a transgenic plant having pesticidal activity. The method may include transforming a plant cell with a nucleic acid molecule described herein, selecting a plant cell comprising the nucleic acid described herein, and regenerating a transgenic plant from the plant cell comprising the nucleic acid molecule described herein, wherein the transgenic plant expresses the nucleic acid molecule described herein and wherein the transgenic plant has pesticidal activity.

In one aspect, the present disclosure is directed to a method of protecting a plant from pest infestation related damage. The method may include introducing to the plant a nucleic acid molecule described herein, wherein the plant expresses the nucleic acid molecule and wherein the resulting polypeptide has pesticidal activity.

The plants or transgenic plants described herein may be protected from infection by plant pests including, but not limited to, fall armyworm (Spodoptera frugiperda) (FAW), corn earworm (Helicoverpa zea) (CEW), European corn borer (Ostrinia nubilalis), cotton boll worm (Helicoverpa armigera), black cutworm (Agrotis ipsilon), lesser cornstalk borer (Elasmopalpus lignosellus), Asian corn borer (Ostinia furnacalis), southwestern corn borer (Diatraea grandiosella), sugarcane borer (Diatraea saccharalis), western bean cutworm (Striacosta albicosta), velvetbean caterpillar (Anticarsia gemmatalis), western corn rootworm (Diabrotica virgifera virgifera), coleopteran species, lepidopteran species, hemipteran species, and combinations thereof.

In one aspect, the present disclosure is directed to a host cell comprising a nucleic acid molecule described herein. Suitable host cells may include prokaryote host cells and eukaryote host cells.

Particularly suitable prokaryote host cells may include archaea and bacteria cells. Particularly suitable eukaryote host cells may include plants and fungi. Suitable host cells may also include microbial cells such as Trichoderma, Aspergillus, Neurospora, Humicola, Penicillium, Fusarium, Thermomonospora, Bacillus, Pseudomonas, Escherichia, Clostridium, Cellulomonas, Streptomyces, Yarrowia, Pichia and Saccharomyces, and microalgal cells belonging to cyanobacterial species. Suitable plant host cells may include dicotyledons and monocotyledons. Suitable dicotyledons may include dicotyledons such as tobacco, cotton, soybean, sunflower, rapeseed, and monocotyledons such as wheat, rice, barley, sorghum, and maize.

In one aspect, the present disclosure is directed to a transgenic plant, a transgenic plant tissue, a transgenic plant cell, or a transgenic plant seed comprising a nucleic acid molecule described herein, and having pesticidal activity.

As described herein, the transformed plant cells, plant parts, or plants may have at least one nucleic acid molecule, nucleic acid construct, expression cassette or vector that encodes a polypeptide having at least 70% sequence identity to any one of SEQ ID NOs: 1-56, at least 75% sequence identity to any one of SEQ ID NOs: 1-56, at least 80% sequence identity to any one of SEQ ID NOs: 1-56, at least 85% sequence identity to any one of SEQ ID NOs: 1-56, at least 90% sequence identity to any one of SEQ ID NOs: 1-56, at least 91% sequence identity to any one of SEQ ID NOs: 1-56, at least 92% sequence identity to any one of SEQ ID NOs: 1-56, at least 93% sequence identity to any one of SEQ ID NOs: 1-56, at least 94% sequence identity to any one of SEQ ID NOs: 1-56, at least 95% sequence identity to any one of SEQ ID NOs: 1-56, at least 96% sequence identity to any one of SEQ ID NOs: 1-56, at least 97% sequence identity to any one of SEQ ID NOs: 1-56, at least 98% sequence identity to any one of SEQ ID NOs: 1-56, at least 99% sequence identity to any one of SEQ ID NOs: 1-56, at least 99.2% sequence identity to any one of SEQ ID NOs: 1-56, at least 99.5% sequence identity to any one of SEQ ID NOs: 1-56, at least 99.8% sequence identity to any one of SEQ ID NOs: 1-56, at least 99.9% sequence identity to any one of SEQ ID NOs: 1-56, or the at least one nucleic acid molecule, nucleic acid construct, expression cassette or vector may encode a polypeptide having the amino acid sequence of any one of SEQ ID NOs: 1-56, wherein the transformed plant cells, transformed plant parts, or transformed plants have pesticidal activity.

The present disclosure also relates to homologs of any of the described insecticidal proteins (e.g., SEQ ID NOs: 1-56), provided that the homologs retain insecticidal or pesticidal activity. Homolog sequences can be isolated from public or private collections and can also be prepared by various conventional methods, such as random mutagenesis, site-directed mutagenesis, gene synthesis, gene engineering, gene editing, or gene shuffling, based on all or a part of the peptide sequences presented in the present disclosure, or using all or part of their coding nucleotide sequences. Such homologs include, for example, deletions, insertions, or substitutions of one or more residues in the amino acid sequence of the protein, or a combination thereof. In some embodiments, a homolog may include a protein having at least 70% sequence identity, at least 75% sequence identity, at least 80% sequence identity, at least 85% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, at least 99.2% sequence identity, at least 99.5% sequence identity, at least 99.8% sequence identity, or at least 99.9% sequence identity to any one of SEQ ID NOs: 1-56.

In some aspects of the present disclosure, a polynucleotide sequence encoding a shuffled insecticidal toxin polypeptide including amino acid substitutions, deletions, insertions, and fragments thereof is disclosed. An insecticidal toxin may have one or more of several domains swapped or shuffled to alter a physical property of the toxin, such as increased efficacy, altered spectrum, reduced plant phytotoxicity, etc.

Pesticidal proteins may be derived from Bacillus thuringiensis (“Bt”), a Gram-positive spore forming soil bacterium. Current commercial pesticidal proteins include Bt Cry (crystal protein), as well as many active insecticidal proteins that lack commercial efficacy, spectrum, or stability. The disclosed embodiments solve some efficacy, spectrum, and/or stability issues in the Gpp(Cry)-like pesticidal protein family of Bt derived insecticidal proteins (see Crickmore, et al., “Bacillus thuringiensis toxin nomenclature” (2011) and Crickmore et al., “A structure-based nomenclature for Bacillus thuringiensis and other bacteria-derived pesticidal proteins” (2021), at bpprc.org).

In addition to the full-length nucleotide sequence of a nucleic acid molecule encoding a polypeptide of any one of SEQ ID NOs: 1-56, the nucleic acid molecule encoding any one of SEQ ID NOs: 1-56 may include a fragment or variant thereof that encodes a polypeptide capable of pesticidal activity. For nucleotide sequences, “fragment” as used herein means a portion of a nucleotide sequence of a nucleic acid molecule, for example, a portion of the nucleotide sequence encoding any one of SEQ ID NOs: 1-56. Fragments of a nucleotide sequence may retain the biological activity of the reference nucleic acid molecule. For example, a nucleic acid molecule encoding less than the entire amino acid sequence disclosed in any one of SEQ ID NOs: 1-56 may be used to encode a protein that retains its pesticidal activity. In other embodiments, fragments of any one of SEQ ID NOs: 1-56 may be used to alter biologically activity of another insecticidal polypeptide sequence or non-insecticidal polypeptide sequence through addition, swapping, or mutating the other insecticidal or non-insecticidal polypeptide with fragments of any one of SEQ ID NOs: 1-56. Alternatively, fragments of a nucleotide sequence can be used as hybridization probes or as an amplification primer. Fragments used as hybridization probes or primers generally do not need to retain biological activity. Thus, fragments of the nucleic acid molecules can be at least about 10, 15, 20, 25, 50, 75, 100, 125, 150, 175, 200, 250, 300, 350, 400, 450, 500, 550, 600, 650, 700, 750, 800, 850 or 900 nucleotides, or up to the number of nucleotides present in a full-length nucleic acid molecule. A biologically active portion (fragment or variant) of the nucleic acid molecule can be prepared by isolating part of the sequence of the nucleic acid molecule, operably linking that fragment to a promoter, expressing the nucleotide sequence encoding the protein, and assessing the amount or activity of the protein.

In some embodiments, the nucleotide sequence or nucleic acid molecule encoding the polypeptide of any one of SEQ ID NOs: 1-56 can also be stacked with nucleotide sequences encoding for agronomic traits such as male sterility, stalk strength, flowering time, other insecticidal proteins, RNA interference transgenes, or transformation technology traits such as cell cycle regulation or gene targeting. These stacked combinations can be created by any method including cross breeding plants by any conventional or TopCross™ methodology, zinc finger nucleases (ZFNs), transcription activator-like effector nucleases (TALENs), clustered regularly interspaced short palindromic repeats (CRISPR), and other genetic transformation or editing. If the traits are stacked by genetically transforming the plants, the nucleotide sequences of interest can be combined at any time and in any order. For example, a transformed plant comprising one or more desired traits can be used as the target to introduce further traits by subsequent transformation. The traits can be introduced simultaneously in a co-transformation protocol with the polynucleotides of interest provided by any combination of transformation cassettes. For example, if two sequences will be introduced, the two sequences can be contained in separate expression cassettes (trans) or contained on the same transformation cassette (cis). Expression of the sequences can be driven by the same promoter or by different promoters.

In one aspect, the present disclosure is directed to a vector that may comprise a nucleic acid molecule encoding a polypeptide having at least 70% sequence identity to any one of SEQ ID NOs: 1-56, at least 75% sequence identity to any one of SEQ ID NOs: 1-56, at least 80% sequence identity to any one of SEQ ID NOs: 1-56, at least 85% sequence identity to any one of SEQ ID NOs: 1-56, at least 90% sequence identity to any one of SEQ ID NOs: 1-56, at least 91% sequence identity to any one of SEQ ID NOs: 1-56, at least 92% sequence identity to any one of SEQ ID NOs: 1-56, at least 93% sequence identity to any one of SEQ ID NOs: 1-56, at least 94% sequence identity to any one of SEQ ID NOs: 1-56, at least 95% sequence identity to any one of SEQ ID NOs: 1-56, at least 96% sequence identity to any one of SEQ ID NOs: 1-56, at least 97% sequence identity to any one of SEQ ID NOs: 1-56, at least 98% sequence identity to any one of SEQ ID NOs: 1-56, at least 99% sequence identity to any one of SEQ ID NOs: 1-56, at least 99.2% sequence identity to any one of SEQ ID NOs: 1-56, at least 99.5% sequence identity to any one of SEQ ID NOs: 1-56, at least 99.8% sequence identity to any one of SEQ ID NOs: 1-56, or at least 99.9% sequence identity to any one of SEQ ID NOs: 1-56.

Suitable vectors are known in the art. Particularly suitable vectors include antibiotic resistance or thermostable antibiotic resistance, or coding for an enzyme that can complement an auxotrophy (natural, such as overcoming the absence of an indispensable amino acid, or engineered, such as URA3-deficient mutants where URA3 is necessary for uracil biosynthesis). Selectable markers include those conferring resistance to antibiotics such as kanamycin (nptll gene), hygromycin (aph IV) spectinomycin (aadA) and gentamycin (aac3 and aacC4) or resistance to herbicides such as glufosinate (bar or pat), dicamba (DMO) and glyphosate (aroA or EPSPS). Selectable markers that allow a direct visual identification of transformation events can also be employed, for example, genes expressing a colored or fluorescent protein such as a luciferase or green fluorescent protein (GFP) or a gene expressing a betaglucuronidase or uidA gene (GUS) for which various chromogenic substrates are known.

In one aspect, the present disclosure is directed to a formulation that may include a recombinant polypeptide having at least 70% sequence identity to any one of SEQ ID NOs: 1-56, at least 75% sequence identity to any one of SEQ ID NOs: 1-56, at least 80% sequence identity to any one of SEQ ID NOs: 1-56, at least 85% sequence identity to any one of SEQ ID NOs: 1-56, at least 90% sequence identity to any one of SEQ ID NOs: 1-56, at least 91% sequence identity to any one of SEQ ID NOs: 1-56, at least 92% sequence identity to any one of SEQ ID NOs: 1-56, at least 93% sequence identity to any one of SEQ ID NOs: 1-56, at least 94% sequence identity to any one of SEQ ID NOs: 1-56, at least 95% sequence identity to any one of SEQ ID NOs: 1-56, at least 96% sequence identity to any one of SEQ ID NOs: 1-56, at least 97% sequence identity to any one of SEQ ID NOs: 1-56, at least 98% sequence identity to any one of SEQ ID NOs: 1-56, at least 99% sequence identity to any one of SEQ ID NOs: 1-56, at least 99.2% sequence identity to any one of SEQ ID NOs: 1-56, at least 99.5% sequence identity to any one of SEQ ID NOs: 1-56, at least 99.8% sequence identity to any one of SEQ ID NOs: 1-56, or at least 99.9% sequence identity to any one of SEQ ID NOs: 1-56, and having pesticidal activity. When applied to a plant, the recombinant polypeptide exhibits pesticidal activity.

Formulations of recombinant polypeptide comprising an acceptable carrier may be in the form of a suspension, a solution, an emulsion, a dusting powder, a dispersible granule, a wettable powder, an emulsifiable concentrate, an aerosol, an impregnated granule, an adjuvant, a coatable paste, encapsulations, or combinations thereof.

Formulations of recombinant polypeptide may include surface-active agents, inert carriers, preservatives, humectants, feeding stimulants, attractants, encapsulating agents, binders, emulsifiers, dyes, UV protectants, buffers, flow agents, fertilizers, solvents, dispersants, wetting agents, tackifiers, micronutrient donors, and combinations thereof.

In one aspect, the present disclosure is directed to a formulation that may include a transformed bacteria comprising a nucleic acid molecule as described herein, and having pesticidal activity. When applied to a plant, the transformed bacteria of the formulation express the nucleic acid molecule and the polypeptide exhibits pesticidal activity.

Formulations of transformed bacteria comprising an acceptable carrier may be in the form of a suspension, a solution, an emulsion, a dusting powder, a dispersible granule, a wettable powder, an emulsifiable concentrate, an aerosol, an impregnated granule, an adjuvant, a coatable paste, encapsulations, or combinations thereof.

Formulations of transformed bacteria may include surface-active agents, inert carriers, preservatives, humectants, feeding stimulants, attractants, encapsulating agents, binders, emulsifiers, dyes, UV protectants, buffers, flow agents, fertilizers, solvents, dispersants, wetting agents, tackifiers, micronutrient donors, and combinations thereof.

Transformed bacteria comprising a nucleic acid molecule as described herein may be used in the same manner that Bacillus thuringiensis strains have previously been used as insecticidal sprays.

The biological activity of interest of the formulations comprising recombinant polypeptide or transformed bacteria is the control of damage-causing plant pests. Such biological activity can be assayed by applying an effective amount of either formulation to a plant having a plant pest, or at risk of being infested by a plant pest, and determining whether the formulation controls the damage-causing plant pests.

In one aspect, the present disclosure is directed to a method for protecting a plant from an insect pest. The method may include expressing in a plant, or a plant cell thereof, a nucleic acid molecule as described herein, wherein the nucleic acid molecule encoding the polypeptide is operably linked to a promoter capable of driving expression in the plant or plant cell thereof, and wherein the encoded polypeptide has pesticidal activity against the insect pest.

Some aspects described herein also encompass antibodies that specifically bind to a chimeric insecticidal protein of the present disclosure. The antibody can optionally be a monoclonal antibody or a polyclonal antisera. In some embodiments, an antibody is selective for the chimeric protein and does not bind to one or more of the parent molecules, and can be used to distinguish the chimeric protein from the parent protein. Such antibodies may be produced using standard immunological techniques for production of polyclonal antisera and, if desired, immortalizing the antibody-producing cells of the immunized host for sources of monoclonal antibody production. The present disclosure also encompasses an insecticidal protein that cross-reacts with an antibody, particularly a monoclonal antibody, raised against one or more of the chimeric insecticidal proteins disclosed herein.

The following experimental examples are offered by way of illustration and not by way of limitation.

### EXAMPLES

### Example 1

In this example, a novel sequence from Dendrothele bispora was identified (SEQ ID NO: 1, as disclosed herein) and found to have insecticidal activity. Further strategic engineering methods were used to create novel insecticidal engineered variants (SEQ ID NOs: 2-56) with increased insecticidal activity, altered spectrum of insecticidal activity, increased durability, or increased stability.

### Example 2

To express the insecticidal polypeptides, the DNA gene coding sequence was synthesized as optimized for expression in E. coli. This sequence was cloned into the pHis Expression Vector (modified version of pRSF-1b (Novagen)) or the pET28a Expression Vector, thus fusing an N-terminal 6×-His TAG coding sequence to the gene. The clone was transformed into E. coli strain BL21(DE3) and grown in an auto-induction medium (OVERNIGHT EXPRESS™ LB medium, EMD Millipore). Following induction, bacterial cells were harvested for recombinant protein purification prior to conducting insect larval activity assays. In some cases, the bacterial culture following induction was used for insect assays.

### Example 3

Insecticidal toxicity bioassays were conducted with transformed bacterial whole cells expressing engineered proteins to evaluate pesticidal efficacy against pests including armyworms, Spodoptera spp, corn earworm, Helicoverpa spp, corn borer, Ostrinia spp, Diatraea spp, Anticarsia spp, and Diabrotica spp.

Corn rootworm, Diabrotica virgifera (CRW), northern corn rootworm, Diabrotica barberi (NCR), and southern corn rootworm, Diabrotica undecimpunctata howardi (SCR) eggs were obtained. Bioassay methods similar to those described by Huynh et al. (2017) and Ludwick et al. (2018) were used to detect insecticidal efficacy. Following incubation, mortality, growth inhibition, and feeding inhibition were assessed.

Fall armyworm, Spodoptera frugiperda (FAW), corn earworm, Helicoverpa zea (CEW), European corn borer, Ostrinia nubilalis (ECB), velvetbean caterpillar, Anticarsia gemmatalis (VBC), southwestern corn borer, Diatraea grandiosella (SWCB), sugarcane borer, Diatraea saccharalis (SCB), soybean looper, Chrysodeixis includens (SBL), beet armyworm, Spodoptera exigua (BAW), southern armyworm, Spodoptera eridania (SAW), tobacco budworm, Chloridia virescens (TBW), western corn rootworm, Diabrotica virgifera virgifera (WCR), and black cutworm, Agrotis ipsilon (BCW) eggs were obtained from a commercial insectary (Benzon Research Inc., Carlisle, PA). Eggs were incubated under controlled temperature and humidity until eclosion. Cry1F R-FAW and Vip3A R-FAW are two field evolved resistant FAW populations that are resistant to the commercial insecticidal proteins Cry1Fa and Vip3A, respectively. Bioassay methods similar to those described by Wang et al. (2019) were used to detect insecticidal efficacy. Following 5 days of incubation, mortality, growth inhibition, and feeding inhibition were assessed (Table 1). While some proteins did not exhibit significant insecticidal activity as tested using this specific diet-overlay bioassay format, other experimental test conditions or bioassays may result in insecticidal activity being measured for those proteins.

TABLE 1
Measured insecticidal activity of whole recombinant
E. coli culture expressing insecticidal proteins.

| | Measured Insecticidal Activity | | | | | | |
|---|---|---|---|---|---|---|---|
| | in Diet-Overlay Bioassay | | | | | | |
| Insecticidal | | | | | | Cry1F | Vip3A |
| Protein | CEW | FAW | ECB | SCB | WCR | R-FAW | R-FAW |
| SEQ ID NO: 1 | + | + | + | + | − | + | + |
| SEQ ID NO: 2 | + | + | NT | + | − | NT | NT |
| SEQ ID NO: 3 | − | − | NT | − | − | NT | NT |
| SEQ ID NO: 4 | + | + | NT | + | − | NT | NT |
| SEQ ID NO: 5 | + | + | NT | + | − | NT | NT |
| SEQ ID NO: 6 | + | + | NT | + | − | NT | NT |
| SEQ ID NO: 7 | + | + | NT | + | − | NT | NT |
| SEQ ID NO: 8 | + | + | NT | + | − | NT | NT |
| SEQ ID NO: 9 | − | − | NT | − | − | NT | NT |
| SEQ ID NO: 10 | + | + | NT | + | − | NT | NT |
| SEQ ID NO: 11 | − | − | NT | − | − | NT | NT |
| SEQ ID NO: 12 | − | − | NT | + | − | NT | NT |
| SEQ ID NO: 13 | − | − | NT | − | − | NT | NT |
| SEQ ID NO: 14 | − | − | NT | − | − | NT | NT |
| SEQ ID NO: 15 | − | − | NT | − | + | NT | NT |
| SEQ ID NO: 16 | − | − | NT | − | − | NT | NT |
| SEQ ID NO: 17 | − | − | NT | − | − | NT | NT |
| SEQ ID NO: 18 | − | − | NT | − | − | NT | NT |
| SEQ ID NO: 19 | − | − | NT | − | − | NT | NT |
| SEQ ID NO: 20 | − | − | NT | − | − | NT | NT |
| SEQ ID NO: 21 | − | − | NT | + | − | NT | NT |
| SEQ ID NO: 22 | − | − | NT | − | + | NT | NT |
| SEQ ID NO: 23 | − | − | NT | − | − | NT | NT |
| SEQ ID NO: 24 | + | + | NT | + | + | NT | NT |
| SEQ ID NO: 25 | + | + | NT | + | − | NT | NT |
| SEQ ID NO: 26 | − | − | NT | − | − | NT | NT |
| SEQ ID NO: 27 | − | − | NT | − | − | NT | NT |
| SEQ ID NO: 28 | − | − | NT | − | − | NT | NT |
| SEQ ID NO: 29 | + | − | NT | + | − | NT | NT |
| SEQ ID NO: 30 | + | − | NT | + | − | NT | NT |
| SEQ ID NO: 31 | + | + | NT | + | − | NT | NT |
| SEQ ID NO: 32 | + | − | NT | − | − | NT | NT |
| SEQ ID NO: 33 | + | + | NT | + | − | NT | NT |
| SEQ ID NO: 34 | + | + | NT | + | − | NT | NT |
| SEQ ID NO: 35 | − | − | NT | − | − | NT | NT |
| SEQ ID NO: 36 | − | − | NT | − | − | NT | NT |
| SEQ ID NO: 37 | − | − | NT | − | − | NT | NT |
| SEQ ID NO: 38 | − | − | NT | − | − | NT | NT |
| SEQ ID NO: 39 | − | − | NT | − | − | NT | NT |
| SEQ ID NO: 40 | − | − | NT | − | − | NT | NT |
| SEQ ID NO: 41 | − | − | NT | − | − | NT | NT |
| SEQ ID NO: 42 | NT | NT | NT | NT | NT | NT | NT |
| SEQ ID NO: 43 | NT | NT | NT | NT | NT | NT | NT |
| SEQ ID NO: 44 | NT | NT | NT | NT | NT | NT | NT |
| SEQ ID NO: 45 | NT | NT | NT | NT | NT | NT | NT |
| SEQ ID NO: 46 | NT | NT | NT | NT | NT | NT | NT |
| SEQ ID NO: 47 | NT | NT | NT | NT | NT | NT | NT |
| SEQ ID NO: 48 | NT | NT | NT | NT | NT | NT | NT |
| SEQ ID NO: 49 | NT | NT | NT | NT | NT | NT | NT |
| SEQ ID NO: 50 | NT | NT | NT | NT | NT | NT | NT |
| SEQ ID NO: 51 | NT | NT | NT | NT | NT | NT | NT |
| SEQ ID NO: 52 | NT | NT | NT | NT | NT | NT | NT |
| SEQ ID NO: 53 | NT | NT | NT | NT | NT | NT | NT |
| SEQ ID NO: 54 | NT | NT | NT | NT | NT | NT | NT |
| SEQ ID NO: 55 | NT | NT | NT | NT | NT | NT | NT |
| SEQ ID NO: 56 | NT | NT | NT | NT | NT | NT | NT |

(+)—indicates that insecticidal activity was measured
(−)—indicates that no insecticidal activity was measured
NT—indicates a condition that was not tested

Agrobacterium-mediated transient expression in Nicotiana benthamiana was used to identify insecticidal activity in planta (Kapila et al., 1997, Schob et al., 1997). Leaf disks constitutively expressing the gene were assayed for reduced feeding damage against susceptible FAW (sFAW), CEW, ECB, SCB, and soybean looper, Chrysodeixis includens (SBL) (Table 2). The sFAW population is a. regenerated population that has never been exposed to insecticide for 20 years and was obtained from a commercial insectary (Benzon Research Inc., Carlisle, PA).

TABLE 2
Measured insecticidal activity of leaf disks expressing insecticidal proteins.

| | | | | | | Phytotoxicity | |
|---|---|---|---|---|---|---|---|
| | | | | | | Early Tox | Late Tox |
| | Leaf Disk Protection | | | | | (after 3 | (after 6 |
| Insecticidal Protein | sFAW | CEW | ECB | SCB | SBL | days) | days) |
| CyclA2 | NT | NT | NT | NT | NT | Y | Y |
| ZsGreen | − | − | − | − | − | NT | NT |
| Cry1Fa | + | NT | + | NT | + | N | N |
| Cry1Ac | NT | + | NT | + | NT | N | N |
| Untransformed | − | − | − | − | − | NT | NT |
| Expression Cassette 1 | − | − | − | − | − | N | N |
| (SEQ ID NO: 1) | | | | | | | |
| Expression Cassette 2 | − | − | − | − | + | N | N |
| (SEQ ID NO: 1) | | | | | | | |
| Expression Cassette 2-CTP | + | + | + | + | + | N | N |
| (SEQ ID NO: 1) | | | | | | | |
| Expression Cassette 3 | − | + | NT | + | NT | N | Y |
| (SEQ ID NO: 1) | | | | | | | |
| Expression Cassette 4 | + | + | NT | + | NT | NT | NT |
| (SEQ ID NO: 1) | | | | | | | |
| Expression Cassette 5 | − | + | NT | + | NT | NT | NT |
| (SEQ ID NO: 1) | | | | | | | |
| Expression Cassette 6 | − | − | NT | + | NT | NT | NT |
| (SEQ ID NO: 1) | | | | | | | |
| Expression Cassette 7 | + | + | NT | + | NT | NT | NT |
| (SEQ ID NO: 1) | | | | | | | |
| Expression Cassette 8 | − | − | NT | + | NT | NT | NT |
| (SEQ ID NO: 1) | | | | | | | |
| Expression Cassette 9 | − | + | NT | + | NT | NT | NT |
| (SEQ ID NO: 1) | | | | | | | |
| SEQ ID NO: 2 | − | + | NT | − | NT | NT | NT |
| SEQ ID NO: 3 | − | − | NT | − | NT | NT | NT |
| SEQ ID NO: 4 | − | + | NT | + | NT | NT | NT |
| SEQ ID NO: 5 | − | + | NT | + | NT | NT | NT |
| SEQ ID NO: 6 | − | − | NT | − | NT | NT | NT |
| SEQ ID NO: 7 | − | − | NT | − | NT | NT | NT |
| SEQ ID NO: 8 | − | + | NT | + | NT | NT | NT |
| SEQ ID NO: 9 | − | − | NT | − | NT | NT | NT |
| SEQ ID NO: 10 | − | − | NT | − | NT | NT | NT |

(+)—indicates that insecticidal activity was measured
(−)—indicates that no insecticidal activity was measured
Y—indicates that phytotoxicity was observed after 3 days (Early Tox) or 6 days (Late Tox)
N—indicates that phytotoxicity was not observed
NT—indicates a condition that was not tested

### Example 5

In this example, different plant binary nucleic acid experimental constructs are produced using various promoters, initiators, introns, enhancers, terminators, upstream regulatory constructs, downstream regulatory constructs, or other regulatory sequence elements that are operably linked to drive expression of a nucleotide sequence encoding any of the pesticidal proteins disclosed herein in a target plant, such as maize cells.

In some examples, these nucleic acid experimental constructs are operably linked to sequences encoding specific targeting peptides, such as a Zea mays chloroplast targeting signal peptide.

Each of the experimental constructs are individually transformed into the maize inbred B104. A minimum of 10 individual, single copy transformation events with intact T-DNAs are produced for each construct. qRT-PCR and western blot analyses are performed on TO leaf material to select transgenic plants showing pesticidal protein expression.

The selected transgenic plants and their progenies from the experimental constructs are grown in greenhouse conditions. Pesticidal activity and efficacy of the different transgenic plants are then evaluated against various pests.

Fall armyworm, Spodoptera frugiperda (FAW) insecticidal efficacy is tested in the greenhouse conditions using a method of artificial infestation of neonate (newly hatched larvae in the 1^{st} larval stage) FAW larvae onto the whorl leaves of the plant and then rating the leaves after the larvae have fed. FAW efficacy assays are deployed in a randomized complete block design of 4 replications of 3 infested plants. Negative (non-transgenic) and positive (transgenic plants expressing reference toxins) controls are utilized as comparators in the root damage assessment. Seeds are counted out and planted in 18 cell flats and placed in a greenhouse bay for germination. The greenhouse bays are set for corn growth with day temperature set to 26-29° C. at 50% RH, and night temperature set to 17-20° C. at 50% RH. The light to dark ratio is 16:8. The seedlings are transplanted into 1-gallon pots at V2 (approximately 14 days). The plants are allowed to grow to V5/V6 growth stage and then each plant is infested with 30 neonate larvae. The neonate larvae are infested in the maize whorl using an inoculator that delivers a 1 mL aliquot of 2040 corn cob grits (used as a carrier) mixed with neonate larvae. Once infested, the larvae feed on the plants for 14 days. When the plants are deemed ready to rate, the Davis Scale for FAW damage is used to select efficacious plants. Analysis of variance (ANOVA, JMP®) is run comparing the transgenic events to the appropriate controls.

Corn earworm, Helicoverpa zea (CEW) insecticidal efficacy is tested in the greenhouse conditions using a method of artificial infestation of neonate CEW (newly hatched larvae in the 1^{st} larval stage) at VT (a few days after each plant is hand pollinated) on the top of the ear in the pollinated silks. CEW efficacy assays are deployed in a randomized complete block design of 4 replications and 3 infested plants. Negative (non-transgenic) and positive (transgenic plants expressing reference toxins) controls are utilized as comparators in the ear damage assessment. Seeds are counted out and planted in 18 cell flats and placed in a greenhouse bay for germination. The greenhouse bays are set for corn growth with day temperature set to 26-29° C. at 50% RH, and night temperature set to 17-20° C. at 50% RH. The light to dark ratio is 16:8. The seedlings are transplanted into 3-gallon pots at V2 (approximately 14 days). After hand pollination, each ear is infested on the pollinated silks with 15 neonate larvae. Once infested, the larvae feed for 21 days. When the ears are deemed ready to rate, each ear is husked back and ear damage is measured in cm² per ear and efficacious plants are selected. Analysis of variance (ANOVA, JMP®) is run comparing the transgenic events to the appropriate controls.

Corn rootworm, Diabrotica virgifera (CRW) insecticidal efficacy is tested in the greenhouse conditions using a method of artificial infestation of eggs into the plant and then rating the roots after the eggs have hatched and the larvae have fed. CRW efficacy assays are deployed in a randomized complete block design of 4 replicates of 3 infested plants. Negative (non-transgenic) and positive (transgenic plants expressing reference toxins) controls are utilized as comparators in the root damage assessment. Seeds are counted out and planted into 32 cell flats and placed in a greenhouse bay for germination. The greenhouse bays are set for corn growth with day temperature set to 26-29° C. at 50% RH, and night temperature set to 17-20° C. at 50% RH. The light to dark ratio is 16:8. The seedlings are transplanted into 1-gallon pots at V2 (approximately 14 days). The plants are allowed to acclimate for approximately 2 days and then are infested with CRW eggs. The eggs are delivered in a 0.16% agar solution at a rate of 500 eggs per mL. Each plant receives 2 mL of egg/agar solution. The solution is delivered in a 1 mL aliquot through a syringe or repeater pipette into each of 2 holes on either side of the plant, approximately 2 inches from the base of the plant and 2 inches deep. The eggs hatch after infestation in approximately 12 days. Once hatched, the larvae feed for approximately 17-21 days. Plants are checked throughout the feeding cycle to monitor feeding progress and proper time to rate. When the plants are determined to be ready, the plants are removed from the greenhouse and washed and rated in a root processing area of the greenhouse complex. The roots are rated using the Iowa State NIS corn injury scale. Analysis of variance (ANOVA, JMP®) is run comparing the transgenic events to the appropriate controls.

European corn borer, Ostrinia nubilalis (ECB) insecticidal efficacy is tested in the greenhouse using a method of artificial infestation of neonate ECB (newly hatched larvae in the 1^{st} larval stage) at VT/R1 above primary ear and below the secondary ear and then rating the internal stalk and ear shank damage after the larvae have fed. ECB efficacy assays are deployed in a randomized complete block design of 4 replications of 3 infested plants. Negative (non-transgenic) and positive (transgenic plants expressing reference toxins) controls are utilized as comparators in stalk and ear shank damage assessment. Seeds are counted out and planted in 18 cell flats and placed in a greenhouse bay for germination. The greenhouse bays are set for corn growth with day temperature set to 26-29° C. at 50% RH, and night temperature set to 17-20° C. at 50% RH. The light to dark ratio is 16:8. The seedlings are transplanted into 3-gallon pots at V2 (approximately 14 days). The plants are allowed to grow to VT/R1 growth stage and then each plant is infested one node above the primary ear and one node below the secondary ear with 50 neonate larvae (100 neonate larvae total). The neonate larvae are infested at the proper nodes where the leaf meets the stalk using an inoculator that delivers a 1 mL aliquot of 2040 corn cob grits (used as a carrier) mixed with neonate larvae. Once infested, the larvae feed for 45-60 days. When the plants are deemed ready to rate, each stalk and ear shank is split and the internal damage is measured in cm and efficacious plants are selected. Analysis of variance (ANOVA, JMP®) is run comparing the transgenic events to the appropriate controls.

In view of the above, it will be seen that several advantages of the disclosure are achieved, and other advantageous results attained. As various changes could be made in the above methods without departing from the scope of the disclosure, it is intended that all matter contained in the above description and shown in the accompanying drawings shall be interpreted as illustrative and not in a limiting sense.

The foregoing description of the specific aspects will so fully reveal the general nature of the invention that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

For reasons of completeness, various aspects of the inventions described herein are set out in the following numbered clauses:
Clause 1. A method of protecting a plant from infection by a plant pathogen or pest, the method comprising:
transforming the plant with a nucleic acid molecule encoding a polypeptide having at least 80% sequence identity to any one of SEQ ID NOs: 1-56 to generate a transformed plant expressing the polypeptide, wherein said polypeptide has pesticidal activity against the plant pathogen or pest; and
regenerating the transformed plant expressing the polypeptide.
Clause 2. The method of clause 1, wherein the polypeptide has at least 95% sequence identity to any one of SEQ ID NOs: 1-56.
Clause 3. The method of clause 1 or 2, wherein the polypeptide is any one of SEQ ID NOs: 1-56.
Clause 4. The method of any one of clauses 1-3, wherein the plant pathogen or pest is selected from the group consisting of fall armyworm (Spodoptera frugiperda), corn earworm (Helicoverpa zea), European corn borer (Ostrinia nubilalis), cotton boll worm (Helicoverpa armigera), black cutworm (Agrotis ipsilon), lesser cornstalk borer (Elasmopalpus lignosellus), Asian corn borer (Ostinia furnacalis), southwestern corn borer (Diatraea grandiosella), sugarcane borer (Diatraea saccharalis), western bean cutworm (Striacosta albicosta), velvetbean caterpillar (Anticarsia gemmatalis), corn rootworm (Diabrotica virgifera), southern corn rootworm (Diabrotica undecimpunctata howardi), northern corn rootworm (Diabrotica barberi), soybean looper (Chrysodeixis includens), tobacco budworm (Chloridia virescens), beet armyworm (Spodoptera exigua), southern armyworm (Spodoptera eridania), and combinations thereof.
Clause 5. A transformed plant, seed, or plant part comprising a recombinant nucleic acid molecule encoding a polypeptide having at least 80% sequence identity to any one of SEQ ID NOs: 1-56 stably incorporated into a genome of the transformed plant, seed, or plant part, wherein the transformed plant, seed, or plant part stably expresses the polypeptide, and wherein the polypeptide has pesticidal activity against a plant pathogen or pest.
Clause 6. The transformed plant, seed, or plant part of clause 5, wherein the polypeptide has at least 95% sequence identity to any one of SEQ ID NOs: 1-56.
Clause 7. The transformed plant, seed, or plant part of clause 5 or 6, wherein the polypeptide is any one of SEQ ID NOs: 1-56.
Clause 8. The transformed plant, seed, or plant part of any one of clauses 5-7, wherein the transformed plant, seed, or plant part is selected from the group consisting of rice, barley, sorghum, soybean, cotton, maize, rapeseed, sugar cane, tobacco, sunflower, and wheat.
Clause 9. A recombinant nucleic acid molecule comprising a polynucleotide sequence encoding a polypeptide having at least 80% sequence identity to any one of SEQ ID NOs: 1-56, wherein the polypeptide has pesticidal activity against a plant pathogen or pest.
Clause 10. The recombinant nucleic acid molecule of clause 9, wherein the polypeptide has at least 95% sequence identity to any one of SEQ ID NOs: 1-56.
Clause 11. The recombinant nucleic acid molecule of clause 9 or 10, wherein the polypeptide is any one of SEQ ID NOs: 1-56.
Clause 12. The recombinant nucleic acid molecule of any one of clauses 9-11, wherein the polynucleotide sequence encoding the polypeptide is operably linked to one or more promoter sequences.
Clause 13. A vector comprising a recombinant nucleic acid molecule comprising a polynucleotide sequence encoding a polypeptide having at least 80% sequence identity to any one of SEQ ID NOs: 1-56, wherein the polypeptide has pesticidal activity against a plant pathogen or pest.
Clause 14. The vector of clause 13, wherein the polypeptide has at least 95% sequence identity to any one of SEQ ID NOs: 1-56.
Clause 15. The vector of clause 13 or 14, wherein the polypeptide is any one of SEQ ID NOs: 1-56.
Clause 16. A transformed host cell comprising a recombinant nucleic acid molecule comprising a polynucleotide sequence encoding a polypeptide having at least 80% sequence identity to any one of SEQ ID NOs: 1-56, wherein the polypeptide has pesticidal activity against a plant pathogen or pest.
Clause 17. The transformed host cell of clause 16, wherein the polypeptide has at least 95% sequence identity to any one of SEQ ID NOs: 1-56.
Clause 18. The transformed host cell of clause 16 or 17, wherein the polypeptide is any one of SEQ ID NOs: 1-56.
Clause 19. A method of treating a plant or plant part against a plant pathogen or pest, the method comprising:
applying to the plant or plant part an effective amount of at least one polypeptide having at least 80% sequence identity to any one of SEQ ID NOs: 1-56, wherein the polypeptide has pesticidal activity against the plant pathogen or pest.
Clause 20. The method of clause 19, wherein the polypeptide has at least 95% sequence identity to any one of SEQ ID NOs: 1-56.
Clause 21. The method of clause 19 or 20, wherein the polypeptide is any one of SEQ ID NOs: 1-56.
Clause 22. A composition having insecticidal activity against a plant pathogen or pest, the composition comprising an effective amount of at least one polypeptide having at least 80% sequence identity to any one of SEQ ID NOs: 1-56.
Clause 23. The composition of clause 22, wherein the polypeptide has at least 95% sequence identity to any one of SEQ ID NOs: 1-56.
Clause 24. The composition of clause 22 or 23, wherein the polypeptide is any one of SEQ ID NOs: 1-56.

What is claimed is:

1. A transgenic plant, seed, or plant part comprising a recombinant nucleic acid molecule encoding a polypeptide having at least 95% sequence identity to any one of SEQ ID NOs: 1, 2, 4-8, 10, 12, 15, 21-25, and 29-34 stably integrated into a genome of the transgenic plant, seed, or plant part, wherein the transgenic plant, seed, or plant part stably expresses the polypeptide, wherein the polypeptide has pesticidal activity against a plant pathogen or pest, and wherein the recombinant nucleic acid molecule encoding the polypeptide is operably linked to one or more heterologous promoter sequences that direct expression in plant tissue.

2. The transgenic plant, seed, or plant part of claim 1, wherein the plant pathogen or pest comprises fall armyworm (Spodoptera frugiperda), corn earworm (Helicoverpa zea), European corn borer (Ostrinia nubilalis), sugarcane borer (Diatraea saccharalis), western corn rootworm (Diabrotica virgifera virgifera), soybean looper (Chrysodeixis includens), or combinations thereof.

3. The transgenic plant, seed, or plant part of claim 1, wherein the transgenic plant, seed, or plant part comprises rice, barley, sorghum, soybean, cotton, maize, rapeseed, sugar cane, tobacco, sunflower, or wheat.

4. The transgenic plant, seed, or plant part of claim 1, wherein the polypeptide is any one of SEQ ID NOs: 1, 2, 4-8, 10, 12, 15, 21-25, and 29-34 or a variant thereof having pesticidal activity and at least 95% sequence identity thereto.

5. The transgenic plant, seed, or plant part of claim 1, wherein the recombinant nucleic acid molecule encoding the polypeptide further comprises one or more heterologous transcriptional enhancers or introns that increase expression in plant cells.

6. A recombinant nucleic acid molecule comprising a polynucleotide sequence encoding a polypeptide having at least 95% sequence identity to any one of SEQ ID NOs: 1, 2, 4-8, 10, 12, 15, 21-25, and 29-34, wherein the polypeptide has pesticidal activity against a plant pathogen or pest, and wherein the polynucleotide sequence encoding the polypeptide is operably linked to one or more heterologous promoter sequences that direct expression in plant tissue.

7. The recombinant nucleic acid molecule of claim 6, wherein the plant pathogen or pest comprises fall armyworm (Spodoptera frugiperda), corn earworm (Helicoverpa zea), European corn borer (Ostrinia nubilalis), sugarcane borer (Diatraea saccharalis), western corn rootworm (Diabrotica virgifera virgifera), soybean looper (Chrysodeixis includens), or combinations thereof.

8. The recombinant nucleic acid molecule of claim 6, wherein the plant tissue comprises rice, barley, sorghum, soybean, cotton, maize, rapeseed, sugar cane, tobacco, sunflower, or wheat.

9. The recombinant nucleic acid molecule of claim 6, wherein the polypeptide is any one of SEQ ID NOs: 1, 2, 4-8, 10, 12, 15, 21-25, and 29-34 or a variant thereof having pesticidal activity and at least 95% sequence identity thereto.

10. The recombinant nucleic acid molecule of claim 6, wherein the polynucleotide sequence encoding the polypeptide comprises one or more codon-optimization modifications enhancing expression in monocots or dicots.

11. A vector comprising the recombinant nucleic acid molecule of claim 6.

12. A cell comprising the recombinant nucleic acid molecule of claim 6.

13. The cell of claim 12, wherein the cell is a prokaryotic cell or a eukaryotic cell.

14. A vector comprising a recombinant nucleic acid molecule comprising a polynucleotide sequence encoding a polypeptide having at least 95% sequence identity to any one of SEQ ID NOs: 1, 2, 4-8, 10, 12, 15, 21-25, and 29-34, wherein the polypeptide has pesticidal activity against a plant pathogen or pest, and wherein the polynucleotide sequence encoding the polypeptide is operably linked to one or more heterologous promoter sequences that direct expression in plant tissue.

15. The vector of claim 14, wherein the plant pathogen or pest comprises fall armyworm (Spodoptera frugiperda), corn earworm (Helicoverpa zea), European corn borer (Ostrinia nubilalis), sugarcane borer (Diatraea saccharalis), western corn rootworm (Diabrotica virgifera virgifera), soybean looper (Chrysodeixis includens), or combinations thereof.

16. The vector of claim 14, wherein the plant tissue comprises rice, barley, sorghum, soybean, cotton, maize, rapeseed, sugar cane, tobacco, sunflower, or wheat.

17. A cell comprising the vector of claim 14.

18. The cell of claim 17, wherein the cell is a prokaryotic cell or a eukaryotic cell.

19. The vector of claim 14, wherein the polypeptide is any one of SEQ ID NOs: 1, 2, 4-8, 10, 12, 15, 21-25, and 29-34.

20. A transformed host cell comprising a recombinant nucleic acid molecule comprising a polynucleotide sequence encoding a polypeptide having at least 95% sequence identity to any one of SEQ ID NOs: 1, 2, 4-8, 10, 12, 15, 21-25, and 29-34, wherein the polypeptide has pesticidal activity against a plant pathogen or pest, and wherein the polynucleotide sequence encoding the polypeptide is operably linked to one or more heterologous promoter sequences that direct expression in plant tissue.

21. The transformed host cell of claim 20, wherein the plant pathogen or pest comprises fall armyworm (Spodoptera frugiperda), corn earworm (Helicoverpa zea), European corn borer (Ostrinia nubilalis), sugarcane borer (Diatraea saccharalis), western corn rootworm (Diabrotica virgifera virgifera), soybean looper (Chrysodeixis includens), or combinations thereof.

22. The transformed host cell of claim 20, wherein the plant tissue comprises rice, barley, sorghum, soybean, cotton, maize, rapeseed, sugar cane, tobacco, sunflower, or wheat.

23. The transformed host cell of claim 20, wherein the transformed host cell is a prokaryote host cell or a eukaryote host cell.

24. The transformed host cell of claim 20, wherein the polypeptide is any one of SEQ ID NOs: 1, 2, 4-8, 10, 12, 15, 21-25, and 29-34.


SEQUENCE LISTING (56 sequences)

SEQ ID NO: 1 - protein, 285 aa, Dendrothele bispora
MSLGPRTTWNDLAHLGVSRHRAYITLNPLRGGTMQDLNDALVNDSYTDNFGWFCYNSILDSPTLVSNSLVPNTTSEVSWSYDNTNNNQSHSETWTEEWEERESATVSITQRSSITLESTINLYNVASTSLSFSIDTEDNSTKSSESIRRMTRSWTMNVGPGERLSLIRNITRSTGTATYTNRYGLHPSHDIIGTKGRRWNNHFYWGYHAKDVFGPLTGSMRFEAHSTITSFTFTLDRVTADGRHTVEPLPLPSGSRMGGIIEGAIPGIDDKDPEEDDPEERQVRA

SEQ ID NO: 2 - protein, 285 aa, synthetic construct
MSLGPRTTWNDLAHLGVSRHRAYITLNPLRGGTMKDLNDALVNDSYTDNFGWFCYNSILDSPTLVSNSLVPNTTSEVSWSYDNTNNNQSHSETWTEEWEERESATVSITQRSSITLESTINLYNVASTSLSFSIDTEDNSTKSSESIRRMTRSWTMNVGPGERLSLIRNITRSTGTATYTNRYGLHPSHDIIGTKGDRYNGHYYWGYHAKDVFGPLTGSMRFEAHSTITSFTFTLDRVTADGRHTVEPLPLPSGSRMGGIIEGAIPGIDDKDPEEDDPEERQVRA

SEQ ID NO: 3 - protein, 285 aa, synthetic construct
MSLGPRTTWNDLAHLGVSRHRAYITLNPLRGGTMQDLNDALVNDSYTDNFGWFCYNSILDSPTLVSNSLVPNTTSEVSWSYDNTNNNQSHSETWTEEWEERESATVSITQRSSITLESTINLYNVASTSLSFSIDTEDNSTKSSESIRRMTRSWTMNVGPGERLSLIRNITRSTGTATYTNRYGLHPSHDIIGTFNPPHNNHFYWGYHAKDVFGPLTGSMRFEAHSTITSFTFTLDRVTADGRHTVEPLPLPSGSRMGGIIEGAIPGIDDKDPEEDDPEERQVRA

SEQ ID NO: 4 - protein, 285 aa, synthetic construct
MSLGPRTTWNDLAHLGVSRHRAYITLNPLRGGTMQDLNDALVNDSYTDNFGWFCYNSILDSPTLVSNSLVPNTTSEVSWSYDNTNNNQSHSETWTEEWEERESATVSITQRSSITLESTINLYNVASTSLSFSIDTEDNSTKSSESIRRMTRSWTMNVGPGERLSLIRNITRSTGTATYTNRYGLHPSHDIIGTKGDRYNNHFYWGYHAKDVFGPLTGSMRFEAHSTITSFTFTLDRVTADGRHTVEPLPLPSGSRMGGIIEGAIPGIDDKDPEEDDPEERQVRA

SEQ ID NO: 5 - protein, 285 aa, synthetic construct
MSLGPRTTWNDLAHLGVSRHRAYITLNPLRGGTMQDLNDALVNDSYTDNFGWFCYNSILDSPTLVSNSLVPNTTSEVSWSYDNTNNNQSHSETWTEEWEERESATVSITQRSSITLESTINLYNVASTSLSFSIDTEDNSTKSSESIRRMTRSWTMNVGPGERLSLIRNITRSTGTATYTNRYGLHPSHDIIGTKGRRWNNHHYWGYHAKDVFGPLTGSMRFEAHSTITSFTFTLDRVTADGRHTVEPLPLPSGSRMGGIIEGAIPGIDDKDPEEDDPEERQVRA

SEQ ID NO: 6 - protein, 285 aa, synthetic construct
MSLGPRTTWNDLAHLGVSRHRAYITLNPLRGGTMQDLNDALVNDSYTDNFGWFCYNSILDSPTLVSNSLVPNTTSEVSWSYDNTNNNQSHSETWTEEWEERESATVSITQRSSITLESTINLYNVASTSLSFSIDTEDNSTKSSESIRRMTRSWTMNVGPGERLSLIRNITRSTGTATYTNRYGLHPSHDIIGTFNPPHNNHHYWGYHAKDVFGPLTGSMRFEAHSTITSFTFTLDRVTADGRHTVEPLPLPSGSRMGGIIEGAIPGIDDKDPEEDDPEERQVRA

SEQ ID NO: 7 - protein, 285 aa, synthetic construct
MSLGPRTTWNDLAHLGVSRHRAYITLNPLRGGTMQDLNDALVNDSYTDNFGWFCYNSILDSPTLVSNSLVPNTTSEVSWSYDNTNNNQSHSETWTEEWEERESATVSITQRSSITLESTINLYNVASTSLSFSIDTEDNSTKSSESIRRMTRSWTMNVGPGERLSLIRNITRSTGTATYTNRYGLHPSHDIIGTKGDRYNNHHYWGYHAKDVFGPLTGSMRFEAHSTITSFTFTLDRVTADGRHTVEPLPLPSGSRMGGIIEGAIPGIDDKDPEEDDPEERQVRA

SEQ ID NO: 8 - protein, 285 aa, synthetic construct
MSLGPRTTWNDLAHLGVSRHRAYITLNPLRGGTMQDLNDALVNDSYTDNFGWFCYNSILDSPTLVSNSLVPNTTSEVSWSYDNTNNNQSHSETWTEEWEERESATVSITQRSSITLESTINLYNVASTSLSFSIDTEDNSTKSSESIRRMTRSWTMNVGPGERLSLIRNITRSTGTATYTNRYGLHPSHDIIGTKGRRWNNHYYWGYHAKDVFGPLTGSMRFEAHSTITSFTFTLDRVTADGRHTVEPLPLPSGSRMGGIIEGAIPGIDDKDPEEDDPEERQVRA

SEQ ID NO: 9 - protein, 285 aa, synthetic construct
MSLGPRTTWNDLAHLGVSRHRAYITLNPLRGGTMQDLNDALVNDSYTDNFGWFCYNSILDSPTLVSNSLVPNTTSEVSWSYDNTNNNQSHSETWTEEWEERESATVSITQRSSITLESTINLYNVASTSLSFSIDTEDNSTKSSESIRRMTRSWTMNVGPGERLSLIRNITRSTGTATYTNRYGLHPSHDIIGTFNPPHNNHYYWGYHAKDVFGPLTGSMRFEAHSTITSFTFTLDRVTADGRHTVEPLPLPSGSRMGGIIEGAIPGIDDKDPEEDDPEERQVRA

SEQ ID NO: 10 - protein, 285 aa, synthetic construct
MSLGPRTTWNDLAHLGVSRHRAYITLNPLRGGTMQDLNDALVNDSYTDNFGWFCYNSILDSPTLVSNSLVPNTTSEVSWSYDNTNNNQSHSETWTEEWEERESATVSITQRSSITLESTINLYNVASTSLSFSIDTEDNSTKSSESIRRMTRSWTMNVGPGERLSLIRNITRSTGTATYTNRYGLHPSHDIIGTKGDRYNNHYYWGYHAKDVFGPLTGSMRFEAHSTITSFTFTLDRVTADGRHTVEPLPLPSGSRMGGIIEGAIPGIDDKDPEEDDPEERQVRA

SEQ ID NO: 11 - protein, 285 aa, synthetic construct
MSLGPKTTWNELAHLGVSKHKAYITLNPLKGGTMQELNEALVNESYTENFGWFCYNSILESPTLVSNSLVPNTTSDVSWSYENTNNNQSHSDTWTDDWDDKDSATVSITQKSSITLDSTINLYNVASTSLSFSIETDENSTRSSDSIKKMTKSWTMNVGPGDKLSLIKNITKSTGTATYTNKYGLHPSHEIIGTRGKKWNNHFYWGYHAREVFGPLTGSMKFDAHSTITSFTFTLEKVTAEGKHTVDPLPLPSGSKMGGIIDGAIPGIEEREPDDEEPDDKQVKA

SEQ ID NO: 12 - protein, 285 aa, synthetic construct
MSLGPKTTWNELAHLGVSKHKAYITLNPLKGGTMQELNEALVNESYTENFGWFCYNSILESPTLVSNSLVPNTTSDVSWSYENTNNNQSHSDTWTDDWDDKDSATVSITQKSSITLESTINLYNVASTSLSFSIDTDENSTRSSDSIKKMTKSWTMNVGPGDKLSLIKNITKSTGTATYTNKYGLHPSHEIIGTRGKKWNNHFYWGYHAREVFGPLTGSMKFDAHSTITSFTFTLEKVTAEGKHTVDPLPLPSGSKMGGIIDGAIPGIEEREPEEEEPDDKQVKA

SEQ ID NO: 13 - protein, 285 aa, synthetic construct
MSLGPRTTWNDLAHLGVSRHRAYITLNPLRGGTMQDLNDALVNDSYTDNFGWFCYNSILESPTLVSNSLVPNTTSDVSWSYENTNNNQSHSDTWTDDWDDKDSATVSITQKSSITLDSTINLYNVASTSLSFSIETDENSTRSSDSIKKMTKSWTMNVGPGDKLSLIKNITKSTGTATYTNKYGLHPSHDIIGTKGRRWNNHFYWGYHAKDVFGPLTGSMKFDAHSTITSFTFTLEKVTAEGKHTVDPLPLPSGSKMGGIIDGAIPGIEEREPEEEEPDDKQVKA

SEQ ID NO: 14 - protein, 285 aa, synthetic construct
MSLGPRTTWNDLAHLGVSRHRAYITLNPLRGGTMQDLNDALVNDSYTDNFGWFCYNSILESPTLVSNSLVPNTTSDVSWSYENTNNNQSHSDTWTDDWDDKDSATVSITQKSSITLESTINLYNVASTSLSFSIDTDENSTRSSDSIKKMTKSWTMNVGPGDKLSLIKNITKSTGTATYTNKYGLHPSHDIIGTKGRRWNNHFYWGYHAKDVFGPLTGSMKFDAHSTITSFTFTLEKVTAEGKHTVDPLPLPSGSKMGGIIDGAIPGIEEREPEEEEPDDKQVKA

SEQ ID NO: 15 - protein, 285 aa, synthetic construct
MSLGPRTTWNELAHLGVSRHRAYITLNPLRGGTMQELNEALVNESYTENFGWFCYNSILESPTLVSNSLVPNTTSDVSWSYENTNNNQSHSDTWTDDWDDRDSATVSITQRSSITLDSTINLYNVASTSLSFSIETDENSTKSSDSIRRMTRSWTMNVGPGDRLSLIRNITRSTGTATYTNRYGLHPSHEIIGTKGRRWNNHFYWGYHAKEVFGPLTGSMRFDAHSTITSFTFTLERVTAEGRHTVDPLPLPSGSRMGGIIDGAIPGIEEKEPDDEEPDDRQVRA

SEQ ID NO: 16 - protein, 285 aa, synthetic construct
MSLGPRTTWNELAHLGVSRHRAYITLNPLRGGTMQELNEALVNESYTENFGWFCYNSILESPTLVSNSLVPNTTSDVSWSYENTNNNQSHSDTWTDDWDDRDSATVSITQRSSITLESTINLYNVASTSLSFSIDTDENSTKSSDSIRRMTRSWTMNVGPGDRLSLIRNITRSTGTATYTNRYGLHPSHEIIGTKGRRWNNHFYWGYHAKEVFGPLTGSMRFDAHSTITSFTFTLERVTAEGRHTVDPLPLPSGSRMGGIIDGAIPGIEEKEPEEEEPDDRQVRA

SEQ ID NO: 17 - protein, 285 aa, synthetic construct
MSLGPRTTWNDLAHLGVSRHRAYITLNPLRGGTMQDLNDALVNDSYTDNFGWFCYNSILESPTLVSNSLVPNTTSDVSWSYENTNNNQSHSDTWTDDWDDRDSATVSITQRSSITLDSTINLYNVASTSLSFSIETDENSTKSSDSIRRMTRSWTMNVGPGDRLSLIRNITRSTGTATYTNRYGLHPSHDIIGTKGRRWNNHFYWGYHAKDVFGPLTGSMRFDAHSTITSFTFTLERVTAEGRHTVDPLPLPSGSRMGGIIDGAIPGIEEKEPEEEEPDDRQVRA

SEQ ID NO: 18 - protein, 285 aa, synthetic construct
MSLGPRTTWNDLAHLGVSRHRAYITLNPLRGGTMQDLNDALVNDSYTDNFGWFCYNSILESPTLVSNSLVPNTTSDVSWSYENTNNNQSHSDTWTDDWDDRDSATVSITQRSSITLESTINLYNVASTSLSFSIDTDENSTKSSDSIRRMTRSWTMNVGPGDRLSLIRNITRSTGTATYTNRYGLHPSHDIIGTKGRRWNNHFYWGYHAKDVFGPLTGSMRFDAHSTITSFTFTLERVTAEGRHTVDPLPLPSGSRMGGIIDGAIPGIEEKEPEEEEPDDRQVRA

SEQ ID NO: 19 - protein, 285 aa, synthetic construct
MSLGPKTTWNDLAHLGVSKHKAYITLNPLKGGTMQDLNDALVNDSYTDNFGWFCYNSILDSPTLVSNSLVPNTTSEVSWSYDNTNNNQSHSETWTEEWEEKESATVSITQKSSITLESTINLYNVASTSLSFSIDTEDNSTRSSESIKKMTKSWTMNVGPGEKLSLIKNITKSTGTATYTNKYGLHPSHDIIGTRGKKWNNHFYWGYHARDVFGPLTGSMKFEAHSTITSFTFTLDKVTADGKHTVEPLPLPSGSKMGGIIEGAIPGIDDRDPEEDDPEEKQVKA

SEQ ID NO: 20 - protein, 285 aa, synthetic construct
MSLGPKTTWNDLAHLGVSKHKAYITLNPLKGGTMQDLNDALVNDSYTDNFGWFCYNSILDSPTLVSNSLVPNTTSEVSWSYDNTNNNQSHSETWTEEWEEKESATVSITQKSSITLESTINLYNVASTSLSFSIDTEDNSTKSSESIKKMTKSWTMNVGPGEKLSLIKNITKSTGTATYTNKYGLHPSHDIIGTKGKKWNNHFYWGYHAKDVFGPLTGSMKFEAHSTITSFTFTLDKVTADGKHTVEPLPLPSGSKMGGIIEGAIPGIDDKDPEEDDPEEKQVKA

SEQ ID NO: 21 - protein, 285 aa, synthetic construct
MSLGPRTTWNELAHLGVSRHRAYITLNPLRGGTMQELNEALVNESYTENFGWFCYNSILESPTLVSNSLVPNTTSEVSWSYENTNNNQSHSETWTEEWEERESATVSITQRSSITLESTINLYNVASTSLSFSIETEENSTKSSESIRRMTRSWTMNVGPGERLSLIRNITRSTGTATYTNRYGLHPSHEIIGTKGRRWNNHFYWGYHAKEVFGPLTGSMRFEAHSTITSFTFTLERVTAEGRHTVEPLPLPSGSRMGGIIEGAIPGIEEKEPEEEEPEERQVRA

SEQ ID NO: 22 - protein, 285 aa, synthetic construct
MSLGPRTTWNDLAHLGVSRHRAYITLNPLRGGTMQDLNDALVNDSYTDNFGWFCYNSILDSPTLVSNSLVPNTTSDVSWSYDNTNNNQSHSDTWTDDWDDRDSATVSITQRSSITLDSTINLYNVASTSLSFSIDTDDNSTKSSDSIRRMTRSWTMNVGPGDRLSLIRNITRSTGTATYTNRYGLHPSHDIIGTKGRRWNNHFYWGYHAKDVFGPLTGSMRFDAHSTITSFTFTLDRVTADGRHTVDPLPLPSGSRMGGIIDGAIPGIDDKDPDDDDPDDRQVRA

SEQ ID NO: 23 - protein, 285 aa, synthetic construct
MSLGPRTTWNDLAHLGVSRHRAYITLNPLRGGTMQDLNDALVNDSYTDNFGWFCYNSILDSPTLVSNSLVPNTTSEVSWSYDNTNNNQSHSETWTEEWEEKESATVSITQKSSITLESTINLYNVASTSLSFSIDTEDNSTRSSESIKKMTKSWTMNVGPGEKLSLIKNITKSTGTATYTNKYGLHPSHDIIGTKGRRWNNHFYWGYHAKDVFGPLTGSMKFEAHSTITSFTFTLDKVTADGKHTVEPLPLPSGSKMGGIIEGAIPGIDDRDPEEDDPEEKQVKA

SEQ ID NO: 24 - protein, 285 aa, synthetic construct
MSLGPRTTWNDLAHLGVSRHRAYITLNPLRGGTMQDLNDALVNDSYTDNFGWFCYNSILDSPTLVSNSLVPNTTSEVSWSYDNTNNNQSHSETWTEEWEERESATVSITQRSSITLESTINLYNVASTSLSFSIDTEDNSTKSSESIRRMTRSWTMNVGPGERLSLIRNITRSTGTATYTNRYGLHPSHDIIGTKGRRWNNHFYWGYHAKDVFGPLTGSMRFEAHSTITSFTFTLDRVTADGRHTVEPLPLPSGSKMGGIIDGAIPGIEEREPDDEEPDDKQVKA

SEQ ID NO: 25 - protein, 285 aa, synthetic construct
MSLGPRTTWNDLAHLGVSRHRAYITLNPLRGGTMQDLNDALVNDSYTDNFGWFCYNSILDSPTLVSNSLVPNTTSEVSWSYDNTNNNQSHSETWTEEWEERESATVSITQRSSITLESTINLYNVASTSLSFSIDTEDNSTKSSESIRRMTRSWTMNVGPGERLSLIRNITRSTGTATYTNRYGLHPSHDIIGTKGRRWNNHFYWGYHAKDVFGPLTGSMRFDAHSTITSFTFTLERVTAEGRHTVDPLPLPSGSRMGGIIDGAIPGIEEKDPDDEEPDDRQVRA

SEQ ID NO: 26 - protein, 285 aa, synthetic construct
MSLGPRTTWNDLAHLGVSRHRAYITLNPLRGGTMQDLNDALVNDSYTDNFGWFCYNSILDSPTLVSNSLVPNTTSEVSWSYENTNNNQSHSDTWTDDWEERESATVSITQRSSITLESTINLYNVASTSLSFSIDTDENSTKSSDSIRRMTRSWTMNVGPGERLSLIRNITRSTGTATYTNRYGLHPSHDIIGTKGRRWNNHFYWGYHAKDVFGPLTGSMRFDAHSTITSFTFTLERVTADGRHTVEPLPLPSGSRMGGIIEGAIPGIDDKDPDDEEPEERQVRA

SEQ ID NO: 27 - protein, 285 aa, synthetic construct
MSLGPRTTWNDLAHLGVSRHRAYITLNPLRGGTMQDLNDALVNDSYTDNFGWFCYNSILESPTLVSNSLVPNTTSDVSWSYENTNNNQSHSDTWTEEWEERESATVSITQRSSITLESTINLYNVASTSLSFSIDTEDNSTKSSESIRRMTRSWTMNVGPGERLSLIRNITRSTGTATYTNRYGLHPSHDIIGTKGRRWNNHFYWGYHAKDVFGPLTGSMRFDAHSTITSFTFTLERVTAEGRHTVDPLPLPSGSRMGGIIEGAIPGIEDKDPDEEDPEDRQVRA

SEQ ID NO: 28 - protein, 285 aa, synthetic construct
MSLGPRTTWNDLAHLGVSRHRAYITLNPLRGGTMQDLNDALVNDSYTDNFGWFCYNSILESPTLVSNSLVPNTTSDVSWSYENTNNNQSHSDTWTEEWEERESATVSITQRSSITLESTINLYNVASTSLSFSIDTEENSTKSSDSIRRMTRSWTMNVGPGERLSLIRNITRSTGTATYTNRYGLHPSHDIIGTKGRRWNNHFYWGYHAKDVFGPLTGSMRFDAHSTITSFTFTLERVTADGRHTVEPLPLPSGSRMGGIIEGAIPGIEEKDPEEDDPDDRQVRA

SEQ ID NO: 29 - protein, 285 aa, synthetic construct
MSLGPRTTWNDLAHLGVSRHRAYITLNPLRGGTMQDLNDALVNDSYTDNFGWFCYNSILDSPTLVSNSLVPNTTSEVSWSYDNTNNNQSHSETWTEEWEERESATVSITQRSSITLESTINLYNVASTSLSFSIDTEENSTRSSDSIRRMTRSWTMNVGPGERLSLIRNITRSTGTATYTNRYGLHPSHDIIGTKGRRWNNHFYWGYHAKDVFGPLTGSMRFEAHSTITSFTFTLDRVTADGRHTVEPLPLPSGSKMGGIIDGAIPGIEDRDPEEEDPDEKQVKA

SEQ ID NO: 30 - protein, 285 aa, synthetic construct
MSLGPRTTWNDLAHLGVSRHRAYITLNPLRGGTMQDLNDALVNDSYTDNFGWFCYNSILDSPTLVSNSLVPNTTSEVSWSYDNTNNNQSHSETWTEEWEERESATVSITQRSSITLESTINLYNVASTSLSFSIDTEDNSTRSSESIRRMTRSWTMNVGPGEKLSLIKNITKSTGTATYTNRYGLHPSHDIIGTKGRRWNNHFYWGYHAKDVFGPLTGSMKFEAHSTITSFTFTLEKVTADGKHTVDPLPLPSGSRMGGIIEGAIPGIDDRDPEEDDPEERQVRA

SEQ ID NO: 31 - protein, 285 aa, synthetic construct
MSLGPRTTWNDLAHLGVSRHRAYITLNPLRGGTMQDLNDALVNDSYTDNFGWFCYNSILDSPTLVSNSLVPNTTSEVSWSYDNTNNNQSHSETWTEEWEERESATVSITQRSSITLESTINLYNVASTSLSFSIDTEDNSTKSSESIRRMTRSWTMNVGPGERLSLIRNITRSTGTATYTNRYGLHPSHDIIGTKGRRWNNHFYWGYHAKDVFGPLTGSMRFEAHSTITSFTFTLDRVTADGRHTVEPLPLPSGSRMGGIIDGAIPGIEEKEPDDEEPDDRQVRA

SEQ ID NO: 32 - protein, 285 aa, synthetic construct
MSLGPRTTWNDLAHLGVSRHRAYITLNPLRGGTMQDLNDALVNDSYTDNFGWFCYNSILDSPTLVSNSLVPNTTSEVSWSYDNTNNNQSHSETWTEEWEERESATVSITQRSSITLESTINLYNVASTSLSFSIDTEDNSTKSSESIRRMTRSWTMNVGPGERLSLIRNITRSTGTATYTNRYGLHPSHDIIGTKGRRWNNHFYWGYHAKDVFGPLTGSMRFEAHTSISTFSFSLDRVSADGRHSVEPLPLPTGTRMGGIIEGAIPGIDDKDPEEDDPEERQVRA

SEQ ID NO: 33 - protein, 285 aa, synthetic construct
MSLGPRTTWNDLAHLGVSRHRAYITLNPLRGGTMQDLNDALVNDSYTDNFGWFCYNSILDSPTLVSNSLVPNTTSEVSWSYDNTNNNQSHSETWTEEWEERESATVSITQRSSITLESTINLYNVASTSLSFSIDTEDNSTRSSESIRRMTRSWTMNVGPGERLSLIRNITRSTGTATYTNRYGLHPSHDIIGTRGRRWNNHFYWGYHARDVFGPLTGSMRFEAHSTITSFTFTLDRVTADGRHTVEPLPLPSGSRMGGIIEGAIPGIDDRDPEEDDPEERQVRA

SEQ ID NO: 34 - protein, 285 aa, synthetic construct
MSLGPRTTWNDLAHLGVSRHRAYITLNPLRGGTMQDLNDALVNDSYTDNFGWFCYNSILDSPTLVSNSLVPNTTSEVSWSYDNTNNNQSHSETWTEEWEERESATVSITQRSSITLESTINLYNVASTSLSFSIDTEDNSTKSSESIRRMTRSWTMNVGPGERLSLIRNITRSTGTATYTNRYGLHPSHDIIGTKGRRWNNHFYWGYHAKDVFGPLTGSMRFEAHSTITSFTFTLDRVTADGRHTVEPLPLPSGSKMGGIIEGAIPGIDDRDPEEDDPEEKQVKA

SEQ ID NO: 35 - protein, 290 aa, synthetic construct
MSLGPRTTWNDLAHLGLDGDNLFRAVERFRGRWPTGAWVRSPAIAGGVDLYQAYAHQGWRQVVTRLEPISNSLVPNTTSEVSWSYDNTNNNQSHSETWTEEWEERESATVSITQRSSITLESTINLYNVASTSLSFSIDTEDNSTKSSESIRRMTRSWTMNVGPGERLSLIRNITRSTGTATYTNRYGLTGHVAQNFNPPHQGHHFWAHSVNSILQASGPLTGSMRFEAHSTITSFTFTLDRVTADGRHTVEPLPLPSGSRMGGIIEGAIPGIDDKDPEEDDPEERQVRA

SEQ ID NO: 36 - protein, 284 aa, synthetic construct
MSLGPRTTWNDLAHLGWPINEVYKKANANRGGTMKGIGDLSLNSGIAAEYQWWSYNSILDSPTLVSNSLVPNTTSEVSWSYDNTNNNQSHSETWTEEWEERESATVSITQRSSITLESTINLYNVASTSLSFSIDTEDNSTKSSESIRRMTRSWTMNVGPGERLSLIRNITRSTGTATYTNRYGLESGSRVGTKGDRYNGHYYWGMSLNNLLNNPRGSMRFEAHSTITSFTFTLDRVTADGRHTVEPLPLPSGSRMGGIIEGAIPGIDDKDPEEDDPEERQVRA

SEQ ID NO: 37 - protein, 289 aa, synthetic construct
MSLGPRTTWNDRRAFGLDGDNLFRAVERFRGRWPTGAWVRSPAIAGGVDLYQAYAHQGWRQVVTRLEPISNSLVPNTTSEVSWSYDNTNNNQSHSETWTEEWEERESATVSITQRSSITLESTINLYNVASTSLSFSIDTEDNSTKSSESIRRMTRSWTMNVGPGERLSLIRNITRSTGTATYTNRYGLHPSHDIIGTKGRRWNNHFYWGYHAKDVFGPLTGSMRFEAHSTITSFTFTLDRVTADGRHTVEPLPLPSGSRMGGIIEGAIPGIDDKDPEEDDPEERQVRA

SEQ ID NO: 38 - protein, 285 aa, synthetic construct
MSLGPRTTWNDLSRLGWPINEVYKKANANRGGTMKGIGDLSLNSGIAAEYQWWSYNSILDSPTLVSNSLVPNTTSEVSWSYDNTNNNQSHSETWTEEWEERESATVSITQRSSITLESTINLYNVASTSLSFSIDTEDNSTKSSESIRRMTRSWTMNVGPGERLSLIRNITRSTGTATYTNRYGLHPSHDIIGTKGRRWNNHHYWGYHAKDVFGPLTGSMRFEAHSTITSFTFTLDRVTADGRHTVEPLPLPSGSRMGGIIEGAIPGIDDKDPEEDDPEERQVRA

SEQ ID NO: 39 - protein, 289 aa, synthetic construct
MSLGPRTTWNDRRAFGLDGDNLFRAVERFRGRWPTGAWVRSPAIAGGVDLYQAYAHQGWRQVVTRLEPISNSLVPNTTSEVSWSYDNTNNNQSHSETWTEEWEERESATVSITQRSSITLESTINLYNVASTSLSFSIDTEDNSTKSSESIRRMTRSWTMNVGPGERLSLIRNITRSTGTATYTNRYGLHPSHDIIGTKGRRWNNHHYWGYHAKDVFGPLTGSMRFEAHSTITSFTFTLDRVTADGRHTVEPLPLPSGSRMGGIIEGAIPGIDDKDPEEDDPEERQVRA

SEQ ID NO: 40 - protein, 282 aa, synthetic construct
MSLGPRTTERTTFHLGDKQLKDAVKAYFGKSPNDVYLHSPTPWGDLYKKYSWPQVQMILVVQSNSLVPNTTSEVSWSYDNTNNNQSHSETWTEEWEERESATVSITQRSSITLESTINLYNVASTSLSFSIDTEDNSTKSSESIRRMTRSWTMNVGPGERLSLIRNITRSTGTATYTNRYGLHPSHDIIGTKGRRWNNHHYWGYHAKDVFGPLTGSMRFEAHSTITSFTFTLDRVTADGRHTVEPLPLPSGSRMGGIIEGAIPGIDDKDPEEDDPEERQVRA

SEQ ID NO: 41 - protein, 284 aa, synthetic construct
MSLGPRTTWNERTTFHLGDKQLKDAVKAYFGKSPNDVYLHSPTPWGDLYKKYSWPQVQMILDSPTLVSNSLVPNTTSEVSWSYDNTNNNQSHSETWTEEWEERESATVSITQRSSITLESTINLYNVASTSLSFSIDTEDNSTKSSESIRRMTRSWTMNVGPGERLSLIRNITRSTGTATYTNRYGLIGNTAVNYNPTYKDHHFWSLGVAGVMAKGGSMRFEAHSTITSFTFTLDRVTADGRHTVEPLPLPSGSRMGGIIEGAIPGIDDKDPEEDDPEERQVRA

SEQ ID NO: 42 - protein, 285 aa, synthetic construct
MSLGPRTTWNDLAHLGVSRHRAYITLNPLRGGTMQGLNDALVNDSYTDNFGWFCYNSILDSPTLVSNSLVPNTTSEVSWSYDNTNNNQSHSETWTEEWEERESATVSITQRSSITLESTINLYNVASTSLSFSIDTEDNSTKSSESIRRMTRSWTMNVGPGERLSLIRNITRSTGTATYTNRYGLHPSHDIIGTKGRRWNNHFYWGYHAKDVFGPLTGSMRFEAHSTITSFTFTLDRVTADGRHTVEPLPLPSGSRMGGIIEGAIPGIDDKDPEEDDPEERQVRA

SEQ ID NO: 43 - protein, 285 aa, synthetic construct
MSLGPRTTWNDLAHLGVSRHRAYITLNPLRGGTMQDLNDALVNDSYTDNYGWFCYNSILDSPTLVSNSLVPNTTSEVSWSYDNTNNNQSHSETWTEEWEERESATVSITQRSSITLESTINLYNVASTSLSFSIDTEDNSTKSSESIRRMTRSWTMNVGPGERLSLIRNITRSTGTATYTNRYGLHPSHDIIGTKGRRWNNHFYWGYHAKDVFGPLTGSMRFEAHSTITSFTFTLDRVTADGRHTVEPLPLPSGSRMGGIIEGAIPGIDDKDPEEDDPEERQVRA

SEQ ID NO: 44 - protein, 285 aa, synthetic construct
MSLGPRTTWNDLAHLGVSRHRAYITLNPLRGGTMQDLNDALVNDSYTDNYQWFCYNSILDSPTLVSNSLVPNTTSEVSWSYDNTNNNQSHSETWTEEWEERESATVSITQRSSITLESTINLYNVASTSLSFSIDTEDNSTKSSESIRRMTRSWTMNVGPGERLSLIRNITRSTGTATYTNRYGLHPSHDIIGTKGRRWNNHFYWGYHAKDVFGPLTGSMRFEAHSTITSFTFTLDRVTADGRHTVEPLPLPSGSRMGGIIEGAIPGIDDKDPEEDDPEERQVRA

SEQ ID NO: 45 - protein, 285 aa, synthetic construct
MSLGPRTTWNDLAHLGVSRHRAYITLNPLRGGTMQDLNDALVNDSYTDNFGWFCYNSILDSPTLVSNSLVPNTTSEVSWSYDNTNNNQSHSETWTEEWEERESATVSITQRSSITLESTINLYNVASTSLSFSIDTEDNSTKSSESIRRMTRSWTMNVGPGERLSLIRNITRSTGTATYTNRYGLHPSHDIIGTKGRRWNNHYFWFYHAKDVFGPLTGSMRFEAHSTITSFTFTLDRVTADGRHTVEPLPLPSGSRMGGIIEGAIPGIDDKDPEEDDPEERQVRA

SEQ ID NO: 46 - protein, 285 aa, synthetic construct
MSLGPRTTWNDLAHLGVSRHRAYITLNPLRGYTMQDLNDALVNDSYTDNFGWFCYNSILDSPTLVSNSLVPNTTSEVSWSYDNTNNNQSHSETWTEEWEERESATVSITQRSSITLESTINLYNVASTSLSFSIDTEDNSTKSSESIRRMTRSWTMNVGPGERLSLIRNITRSTGTATYTNRYGLHPSHDIIGTKGRRWNNHFYWGYHAKDVFGPLTGSMRFEAHSTITSFTFTLDRVTADGRHTVEPLPLPSGSRMGGIIEGAIPGIDDKDPEEDDPEERQVRA

SEQ ID NO: 47 - protein, 285 aa, synthetic construct
MSLGPRTTWNDLAHLGVSRHRAYITLNPLRGYTMKDLNDALVNDSYTDNYGWFCYNSILDSPTLVSNSLVPNTTSEVSWSYDNTNNNQSHSETWTEEWEERESATVSITQRSSITLESTINLYNVASTSLSFSIDTEDNSTKSSESIRRMTRSWTMNVGPGERLSLIRNITRSTGTATYTNRYGLHPSHDIIGTKGRRWNNHFYWGYHAKDVFGPLTGSMRFEAHSTITSFTFTLDRVTADGRHTVEPLPLPSGSRMGGIIEGAIPGIDDKDPEEDDPEERQVRA

SEQ ID NO: 48 - protein, 285 aa, synthetic construct
MSLGPRTTWNDLAHLGVSRHRAYITLNPLRGGTMQDLNDALVNDSYTDNFQWFCYNSILDSPTLVSNSLVPNTTSEVSWSYDNTNNNQSHSETWTEEWEERESATVSITQRSSITLESTINLYNVASTSLSFSIDTEDNSTKSSESIRRMTRSWTMNVGPGERLSLIRNITRSTGTATYTNRYGLHPSHDIIGTKGRRWNNHFYWGYHAKDVFGPLTGSMRFEAHSTITSFTFTLDRVTADGRHTVEPLPLPSGSRMGGIIEGAIPGIDDKDPEEDDPEERQVRA

SEQ ID NO: 49 - protein, 285 aa, synthetic construct
MSLGPRTTWNDLAHLGVSRHRAYKKLNPLRGGTMQDLNDALVNDSYTDNFGWFCYNSILDSPTLVSNSLVPNTTSEVSWSYDNTNNNQSHSETWTEEWEERESATVSITQRSSITLESTINLYNVASTSLSFSIDTEDNSTKSSESIRRMTRSWTMNVGPGERLSLIRNITRSTGTATYTNRYGLHPSHDIIGTKGRRWNNHFYWGYHAKDVFGPLTGSMRFEAHSTITSFTFTLDRVTADGRHTVEPLPLPSGSRMGGIIEGAIPGIDDKDPEEDDPEERQVRA

SEQ ID NO: 50 - protein, 285 aa, synthetic construct
MSLGPRTTWNDLAHLGVSRHRAYITLNPLRGGTMQDLNDALVNDSYTDEYQWFCYNSILDSPTLVSNSLVPNTTSEVSWSYDNTNNNQSHSETWTEEWEERESATVSITQRSSITLESTINLYNVASTSLSFSIDTEDNSTKSSESIRRMTRSWTMNVGPGERLSLIRNITRSTGTATYTNRYGLHPSHDIIGTKGRRWNNHFYWGYHAKDVFGPLTGSMRFEAHSTITSFTFTLDRVTADGRHTVEPLPLPSGSRMGGIIEGAIPGIDDKDPEEDDPEERQVRA

SEQ ID NO: 51 - protein, 285 aa, synthetic construct
MSLGPRTTWNDLAHLGVSRHRAYITLNPLRGGTMQDLNDALVNDSYTDNFGWFCYNSILDSPTLVSNSLVPNTTSEVSWSYDNTNNNQSHSETWTEEWEERESATVSITQRSSITLESTINLYNVASTSLSFSIDTEDNSTKSSESIRRMTRSWTMNVGPGERLSLIRNITRSTGTATYTNRYGLHPSHDIIGTKGRRWNGHFYWGYHAKDVFGPLTGSMRFEAHSTITSFTFTLDRVTADGRHTVEPLPLPSGSRMGGIIEGAIPGIDDKDPEEDDPEERQVRA

SEQ ID NO: 52 - protein, 285 aa, synthetic construct
MSLGPRTTWNDLAHLGVSRHRAYITLNPLRGGTMHDLNDALVNDSYTDNFGWFCYNSILDSPTLVSNSLVPNTTSEVSWSYDNTNNNQSHSETWTEEWEERESATVSITQRSSITLESTINLYNVASTSLSFSIDTEDNSTKSSESIRRMTRSWTMNVGPGERLSLIRNITRSTGTATYTNRYGLHPSHDIIGTKGRRWNNHFYWGYHAKDVFGPLTGSMRFEAHSTITSFTFTLDRVTADGRHTVEPLPLPSGSRMGGIIEGAIPGIDDKDPEEDDPEERQVRA

SEQ ID NO: 53 - protein, 285 aa, synthetic construct
MSLGPRTTWNDLAHLGVSRHRAYITLNPLRGGTMKDLNDALVNDSYTDNFGWFCYNSILDSPTLVSNSLVPNTTSEVSWSYDNTNNNQSHSETWTEEWEERESATVSITQRSSITLESTINLYNVASTSLSFSIDTEDNSTKSSESIRRMTRSWTMNVGPGERLSLIRNITRSTGTATYTNRYGLHPSHDIIGTKGRRWNNHFYWGYHAKDVFGPLTGSMRFEAHSTITSFTFTLDRVTADGRHTVEPLPLPSGSRMGGIIEGAIPGIDDKDPEEDDPEERQVRA

SEQ ID NO: 54 - protein, 285 aa, synthetic construct
MSLGPRTTWNDLAHLGVSRHRAYITLNPLRGGTMKGLNDALVNDSYTDNYGWFCYNSILDSPTLVSNSLVPNTTSEVSWSYDNTNNNQSHSETWTEEWEERESATVSITQRSSITLESTINLYNVASTSLSFSIDTEDNSTKSSESIRRMTRSWTMNVGPGERLSLIRNITRSTGTATYTNRYGLHPSHDIIGTKGRRWNNHFYWGYHAKDVFGPLTGSMRFEAHSTITSFTFTLDRVTADGRHTVEPLPLPSGSRMGGIIEGAIPGIDDKDPEEDDPEERQVRA

SEQ ID NO: 55 - protein, 285 aa, synthetic construct
MSLGPRTTWNDLAHLGVSRHRAYITLNPLRGGTMQDLNDALVNDSYTDNFGWFCYNSILDSPTLVSNSLVPNTTSEVSWSYDNTNNNQSHSETWTEEWEERESATVSITQRSSITLESTINLYNVASTSLSFSIDTEDNSTKSSESIRRMTRSWTMNVGPGERLSLIRNITRSTGTATYTNRYGLHPSHDIIGTKGRRYNNHFYWGYHAKDVFGPLTGSMRFEAHSTITSFTFTLDRVTADGRHTVEPLPLPSGSRMGGIIEGAIPGIDDKDPEEDDPEERQVRA

SEQ ID NO: 56 - protein, 285 aa, synthetic construct
MSLGPRTTWNDLAHLGVSRHRAYITLNPLRGGTMQDLNDALVNDSYTDNFGWFCYNSILDSPTLVSNSLVPNTTSEVSWSYDNTNNNQSHSETWTEEWEERESATVSITQRSSITLESTINLYNVASTSLSFSIDTEDNSTKSSESIRRMTRSWTMNVGPGERLSLIRNITRSTGTATYTNRYGLHPSHDIIGTKGRRYNGHYYWGYHAKDVFGPLTGSMRFEAHSTITSFTFTLDRVTADGRHTVEPLPLPSGSRMGGIIEGAIPGIDDKDPEEDDPEERQVRA